(19) United States
(12) Patent Application Publication
(10) Pub. No.: US 2009/0069866 A1
(43) Pub. Date: Mar. 12, 2009

(54) IMPLANT TESTER

(75) Inventors: John Michael Farbarik, Castro Valley, CA (US); John C. Potosky, San Jose, CA (US); Anant V. Hegde, Hayward, CA (US); Casidy Domingo, San Mateo, CA (US); Charisse M. Yung, Los Altos Hills, CA (US); Nikhil D. Bhat, Fremont, CA (US); George Yoseung Choi, Redwood City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Pavad Medical, Inc., Fremont, CA (US)

(21) Appl. No.: 12/205,625

(22) Filed: Sep. 5, 2008

Related U.S. Application Data
(63) Continuation-in-part of application No. 11/613,027, filed on Dec. 19, 2006.
Application No. 11/613,027 is a continuation-in-part of application No. 11/355,927, filed on Feb. 15, 2006.
Application No. 11/355,927 is a continuation-in-part of application No. 11/233,493, filed on Sep. 21, 2005.
Application No. 11/233,493 is a continuation-in-part of application No. 10/946,435, filed on Sep. 21, 2004.

(51) Int. Cl. A61N 1/08

(52) U.S. Cl. 607/60

(57) ABSTRACT

An implant testing device and a method of detecting an airway implant are disclosed. The testing device detects the presence of the implant within a patient's body and can be used to determine its location. The testing device also provides an indication of proper function of the implant electronics. A detector circuit of the testing device generates an output signal representative of proximity of the airway implant. A processing circuit receives the output signal and determines proximity of the implant based on one or more detection thresholds. The processing circuit also provides a visual and/or audible alert. In some embodiments, the processing circuit varies the flash rate of one or more light emitting diodes and/or the pitch of an alert tone based on proximity of the implant. Various embodiments of the testing device are adapted for handheld use and can include a handle, elongated portion, and detector element.

### CROSS-REFERENCE

This application is a continuation-in-part of U.S. patent application Ser. No. 11/613,027 (atty. docket no. 026705-000312) filed Dec. 19, 2006 which is a continuation-in-part of U.S. patent application Ser. Nos. 10/946,435, filed Sep. 21, 2004, 11/233,493 filed Sep. 21, 2005, and 11/355,927 filed Feb. 15, 2006, all of which are incorporated herein by reference in their entirety.

### BACKGROUND

Snoring is very common among mammals including humans. Snoring is a noise produced while breathing during sleep due to the vibration of the soft palate and uvula. Not all snoring is bad, except it bothers the bed partner or others near the person who is snoring. If the snoring gets worse overtime and goes untreated, it could lead to apnea.

Those with apnea stop breathing in their sleep, often hundreds of times during the night. Usually apnea occurs when the throat muscles and tongue relax during sleep and partially block the opening of the airway. When the muscles of the soft palate at the base of the tongue and the uvula relax and sag, the airway becomes blocked, making breathing labored and noisy and even stopping it altogether. Sleep apnea also can occur in obese people when an excess amount of tissue in the airway causes it to be narrowed.

In a given night, the number of involuntary breathing pauses or “apneic events” may be as high as 20 to 60 or more per hour. These breathing pauses are almost always accompanied by snoring between apnea episodes. Sleep apnea can also be characterized by choking sensations.

Sleep apnea is diagnosed and treated by primary care physicians, pulmonologists, neurologists, or other physicians with specialty training in sleep disorders. Diagnosis of sleep apnea is not simple because there can be many different reasons for disturbed sleep.

The specific therapy for sleep apnea is tailored to the individual patient based on medical history, physical examination, and the results of polysomnography. Medications are generally not effective in the treatment of sleep apnea. Oxygen is sometimes used in patients with central apnea caused by heart failure. It is not used to treat obstructive sleep apnea.

Nasal continuous positive airway pressure (CPAP) is the most common treatment for sleep apnea. In this procedure, the patient wears a mask over the nose during sleep, and pressure from an air blower forces air through the nasal passages. The air pressure is adjusted so that it is just enough to prevent the throat from collapsing during sleep. The pressure is constant and continuous. Nasal CPAP prevents airway closure while in use, but apnea episodes return when CPAP is stopped or it is used improperly. Many variations of CPAP devices are available and all have the same side effects such as nasal irritation and drying, facial skin irritation, abdominal bloating, mask leaks, sore eyes, and headaches. Some versions of CPAP vary the pressure to coincide with the person's breathing pattern, and other CPAPs start with low pressure, slowly increasing it to allow the person to fall asleep before the full prescribed pressure is applied.

Dental appliances that reposition the lower jaw and the tongue have been helpful to some patients with mild to moderate sleep apnea or who snore but do not have apnea. A dentist or orthodontist is often the one to fit the patient with such a device.

Some patients with sleep apnea may need surgery. Although several surgical procedures are used to increase the size of the airway, none of them is completely successful or without risks. More than one procedure may need to be tried before the patient realizes any benefits. Some of the more common procedures include removal of adenoids and tonsils (especially in children), nasal polyps or other growths, or other tissue in the airway and correction of structural deformities. Younger patients seem to benefit from these surgical procedures more than older patients.

Uvulopalatopharyngoplasty (UPPP) is a procedure used to remove excess tissue at the back of the throat (tonsils, uvula, and part of the soft palate). The success of this technique may range from 30 to 60 percent. The long-term side effects and benefits are not known, and it is difficult to predict which patients will do well with this procedure.

Laser-assisted uvulopalatoplasty (LAUP) is done to eliminate snoring but has not been shown to be effective in treating sleep apnea. This procedure involves using a laser device to eliminate tissue in the back of the throat. Like UPPP, LAUP may decrease or eliminate snoring but not eliminate sleep apnea itself. Elimination of snoring, the primary symptom of sleep apnea, without influencing the condition may carry the risk of delaying the diagnosis and possible treatment of sleep apnea in patients who elect to have LAUP. To identify possible underlying sleep apnea, sleep studies are usually required before LAUP is performed.

Somnoplasty is a procedure that uses RF to reduce the size of some airway structures such as the uvula and the back of the tongue. This technique helps in reducing snoring and is being investigated as a treatment for apnea.

Tracheostomy is used in persons with severe, life-threatening sleep apnea. In this procedure, a small hole is made in the windpipe and a tube is inserted into the opening. This tube stays closed during waking hours and the person breathes and speaks normally. It is opened for sleep so that air flows directly into the lungs, bypassing any upper airway obstruction. Although this procedure is highly effective, it is an extreme measure that is rarely used.

Patients in whom sleep apnea is due to deformities of the lower jaw may benefit from surgical reconstruction. Surgical procedures to treat obesity are sometimes recommended for sleep apnea patients who are morbidly obese. Behavioral changes are an important part of the treatment program, and in mild cases behavioral therapy may be all that is needed. Overweight persons can benefit from losing weight. Even a 10 percent weight loss can reduce the number of apneic events for most patients. Individuals with apnea should avoid the use of alcohol and sleeping pills, which make the airway more likely to collapse during sleep and prolong the apneic periods. In some patients with mild sleep apnea, breathing pauses occur only when they sleep on their backs. In such cases, using pillows and other devices that help them sleep in a side position may be helpful.

Recently, Restore Medical, Inc., Saint Paul, Minn. has developed a new treatment for snoring and apnea, called the Pillar technique. Pillar System is a procedure where 2 or 3 small polyester rod devices are placed in the patient's soft palate. The Pillar System stiffens the palate, reduces vibration of the tissue, and prevents the possible airway collapse. Stiff implants in the soft palate, however, could hinder patient's normal functions like speech, ability to swallow, coughing and sneezing. Protrusion of the modified tissue into the airway is another long-term concern.

As the current treatments for snoring and/or apnea are not effective and have side-effects, there is a need for additional treatment options.

### BRIEF SUMMARY

An implant testing device and a method of detecting an airway implant are disclosed. The testing device detects the presence of the implant within a patient's body and can be used to determine its location. The testing device also provides an indication of proper function of the implant electronics. A detector circuit of the testing device generates an output signal representative of proximity of the airway implant. A processing circuit receives the output signal and determines proximity of the implant based on one or more detection thresholds. The processing circuit also provides a visual and/or audible alert. In some embodiments, the processing circuit varies the flash rate of one or more light emitting diodes and/or the pitch of an alert tone based on proximity of the implant. Various embodiments of the testing device are adapted for handheld use and can include a handle, elongated portion, and detector element.

In one embodiment, the testing device comprises a body having an elongated portion adapted for insertion into the patient's mouth and a handle for manipulating the testing device. A detector is disposed within the body and includes a resonator circuit and a processor. The processor is configured to monitor the resonator circuit and to detect proximity of the implant unit to the testing device. One or more status indicators coupled to the processor are configured to signal proximity of the implant unit to the testing device.

In one embodiment, the processor is configured to deliver a drive signal to the resonator circuit at or near its resonant frequency. The resonator circuit generates an electromagnetic field under the influence of the drive signal. When the testing device nears the implant, the electromagnetic field is disturbed. The disturbance results in a change in resonator current. A proximity detection circuit monitors resonator current and provides an output signal representative of the resonator current to the processor. The processor compares a value of the output signal to one or more detection thresholds and signals proximity of the implant based on the comparison. In one embodiment, the processor signals proximity of the implant by flashing one or more light emitting diodes (LEDs) and generating an audible tone. The flash-rate of the LEDs and pitch of the audible tone can be varied based on the proximity of the implant.

In another embodiment, a method of detecting a palatal implant is disclosed. The method includes generating an electromagnetic field at a testing device and detecting a variation in the electromagnetic field due to proximity of the testing device to the palatal implant. The method also includes indicating the proximity of the testing device to the palatal implant based on the variation of the electromagnetic field. Generating the electromagnetic field can include driving an inductor-capacitor (LC) circuit at approximately a resonant frequency of the inductor-capacitor circuit, and detecting a variation in the electromagnetic field can include detecting a change in the electromagnetic field of the inductor element.

In a further embodiment, a handheld testing device for detecting the presence of a palatal implant unit within a patient's body is disclosed. The handheld testing device includes a body having an elongated portion adapted for insertion into the patient's mouth and a handle portion adapted for manipulating the testing device. The device also includes a detector disposed at a distal end of the elongated portion having a resonator circuit configured to generate an electromagnetic field. A processor is disposed within the handle and configured to detect proximity of the testing device to the implant unit based upon the electromagnetic field. The processor generates an output signal indicative of the proximity. The handheld testing device also includes a user interface circuit including at least one light emitting diode (LED). The user interface circuit is configured to drive the at least one LED based on the output signal.

### BRIEF DESCRIPTION OF THE DRAWINGS

FIG. 1 illustrates one embodiment of the airway implant device.

FIG. 2 illustrates one embodiment of the airway implant device.

FIG. 3 illustrates one embodiment of the airway implant device.

FIG. 4 illustrates one embodiment of the airway implant device.

FIG. 5 illustrates a circuit diagram of an embodiment of the airway implant device.

FIG. 6 illustrates an embodiment of the airway implant device.

FIG. 7 illustrates a sectional view of an embodiment of the electroactive polymer element.

FIG. 8 illustrates a sectional view of an embodiment of the electroactive polymer element.

FIG. 9 illustrates an embodiment of the electroactive polymer element.

FIG. 10 illustrates an embodiment of the electroactive polymer element.

FIG. 11 illustrates an embodiment of the electroactive polymer element.

FIG. 12 illustrates an embodiment of the electroactive polymer element.

FIG. 13 illustrates an embodiment of the electroactive polymer element.

FIG. 14 illustrates an embodiment of the electroactive polymer element.

FIG. 15 illustrates an embodiment of the electroactive polymer element.

FIG. 16 illustrates an embodiment of the electroactive polymer element.

FIG. 17 illustrates an embodiment of the electroactive polymer element.

FIG. 18 illustrates an embodiment of the electroactive polymer element.

FIG. 19 illustrates an embodiment of the electroactive polymer element.

FIG. 20 illustrates an embodiment of the implanted portion of the airway implant device.

FIG. 21 illustrates an embodiment of the airway implant device.

FIG. 22 illustrates an embodiment of the non-implanted portion in the form of a mouth guard.

FIG. 23 illustrates an embodiment of the non-implanted portion in the form of a mouth guard.

FIG. 24 illustrates an embodiment of the non-implanted portion.

FIG. 25 shows a sagittal section through a head of a subject illustrating an embodiment of a method for using the airway implant device.

FIG. 26 illustrates an anterior view of the mouth with see-through mouth roofs to depict an embodiment of a method for using the airway implant device.

FIG. 27 illustrates an anterior view of the mouth with see-through mouth roofs to depict an embodiment of a method for using the airway implant device.

FIG. 28 illustrates an anterior view of the mouth with see-through mouth roofs to depict an embodiment of a method for using the airway implant device.

FIG. 29 illustrates an anterior view of the mouth with see-through mouth roofs to depict an embodiment of a method for using the airway implant device.

FIG. 30 illustrates an embodiment of an inductive coupling system associated with the airway implant device.

FIG. 31 illustrates an embodiment of the airway implant device.

FIG. 32 illustrates an embodiment of the airway implant device.

FIG. 33 illustrates an embodiment in which a patient wears the non-implanted portion of the device on the cheeks.

FIGS. 34A-34B illustrates an embodiment of a method of the invention with the airway implant in the soft palate.

FIGS. 35A-35B illustrates an embodiment of a method of the invention with the airway implants in the soft palate and lateral pharyngeal walls.

FIGS. 36A-36B illustrates an embodiment of a method of the invention with the airway implants in the lateral pharyngeal walls.

FIG. 37 depicts the progression of an apneic event.

FIG. 38 depicts an embodiment of an airway implant device with sensors in the soft palate and laryngeal wall.

FIG. 39 depicts the functioning of an airway implant device with sensors in the soft palate and laryngeal wall.

FIG. 40 depicts an embodiment of an airway implant device with a sensor in the laryngeal wall.

FIG. 41 depicts an example of controller suitable for use with an airway implant device.

FIG. 42 depicts an embodiment of an airway implant device.

FIG. 43 depicts an embodiment of an airway implant device.

FIGS. 44A, 44B, and 44C illustrate terms used in describing the anatomy of a patient and orientation attributes of the invention.

FIG. 45A illustrates an embodiment of the airway implant device.

FIG. 45B illustrates the airway implant device of FIG. 45A, viewed from the anterior side of the implant, looking toward the posterior end, wherein the implant device is implanted in the palate.

FIG. 46A illustrates an embodiment of the airway implant device.

FIG. 46B illustrates the airway implant device of FIG. 46A, viewed from the anterior side of the implant, looking toward the posterior end, wherein the implant device is implanted in the palate.

FIG. 47A illustrates an embodiment of the airway implant device with a T-shaped attachment element.

FIG. 47B illustrates an embodiment of the airway implant device with a perforated attachment element.

FIG. 48 illustrates an embodiment of the airway implant device with saw-blade like directional attachment element.

FIG. 49 illustrates an embodiment of the airway implant device with power connecting element.

FIG. 50 illustrates an embodiment of the airway implant system with both an implantable device and a non-implantable wearable element.

FIG. 51A illustrates an isometric view of the wearable element.

FIG. 51B illustrates a bottom view of the wearable element.

FIG. 52 illustrates a cross-sectional view of the airway implant system in the patient soft palate.

FIGS. 53A-B illustrate one embodiment of an implant testing device.

FIG. 54 is a high-level block diagram of an exemplary testing device.

FIG. 55 is a plot showing aspects of a charge controller according to one embodiment of the present invention.

FIG. 56 is a plot showing aspects of proximity detection according embodiments of the present invention.

FIG. 57 is a block diagram of an exemplary microcontroller such as can be used with the testing device of FIGS. 53-54.

FIG. 58 is a flowchart showing aspects of command processing according to one embodiment of the present invention.

FIG. 59 is a flowchart showing aspects of proximity detection according to one embodiment of the present invention.

FIG. 60 is a flowchart showing aspects of power management according to one embodiment of the present invention.

### DETAILED DESCRIPTION

### Devices and Methods

A first aspect of the invention is a device for the treatment of disorders associated with improper airway patency, such as snoring or sleep apnea. The device comprises of an actuator element to adjust the opening of the airway. In a preferred embodiment, the actuator element comprises of an electroactive polymer (EAP) element. The electroactive polymer element in the device assists in maintaining appropriate airway opening to treat the disorders. Typically, the EAP element provides support for the walls of an airway, when the walls collapse, and thus, completely or partially opens the airway.

The device functions by maintaining energized and non-energized configurations of the EAP element. In preferred embodiments, during sleep, the EAP element is energized with electricity to change its shape and thus modify the opening of the airway. Typically, in the non-energized configuration the EAP element is soft and in the energized configuration is stiffer. The EAP element of the device can have a pre-set non-energized configuration wherein it is substantially similar to the geometry of the patient's airway where the device is implanted.

In some embodiments, the device, in addition to the EAP element, includes an implantable transducer in electrical communication with the EAP element. A conductive lead connects the EAP element and the implantable transducer to the each other. The device of the present invention typically includes a power source in electrical communication with the EAP element and/or the implantable transducer, such as a battery or a capacitor. The battery can be disposable or rechargeable.

Preferred embodiments of the invention include a non-implanted portion, such as a mouthpiece, to control the implanted EAP element. The mouthpiece is typically in conductive or inductive communication with an implantable transducer. In one embodiment, the mouthpiece is a dental retainer with an induction coil and a power source. The dental retainer can further comprise a pulse-width-modulation circuit. When a dental retainer is used it is preferably custom fit for the individual biological subject. If the implantable transducer is in inductive communication, it will typically include an inductive receiver, such as a coil. The implantable transducer can also include a conductive receiver, such as a dental filling, a dental implant, an implant in the oral cavity, an implant in the head or neck region. In one embodiment, the device includes a dermal patch with a coil, circuit and power source, in communication with the implantable transducer. The dermal patch can also include a pulse-width-modulation circuit.

Another aspect of the invention is a method to modulate air flow through airway passages. Such modulation is used in the treatment of diseases such as snoring and sleep apnea. One method of the invention is a method for modulating the airflow in airway passages by implanting in a patient a device comprising an actuator element and controlling the device by energizing the actuator element. The actuator element preferably comprises an electroactive polymer element. The actuator element can be controlled with a mouthpiece inserted into the mouth of the patient. The energizing is typically performed with the use of a power source in electrical communication, either inductive communication or conductive communication, with the actuator element. A transducer can be used to energize the actuator element by placing it in electrical communication with the power source. Depending on the condition being treated, the actuator element is placed in different locations such as soft palate, airway sidewall, uvula, pharynx wall, trachea wall, larynx wall, and/or nasal passage wall.

A preferred embodiment of the device of the present invention comprises an implantable actuator element; an implantable transducer; an implantable lead wire connecting the actuator element and the transducer; a removable transducer; and a removable power source; and wherein the actuator element comprises an electroactive polymer.

Electroactive polymer is a type of polymer that responds to electrical stimulation by physical deformation, change in tensile properties, and/or change in hardness. There are several types of electroactive polymers like dielectric electrostrictive polymer, ion exchange polymer and ion exchange polymer metal composite (IPMC). The particular type of EAP used in the making of the disclosed device can be any of the aforementioned electroactive polymers.

Suitable materials for the electroactive polymer element include, but are not limited to, an ion exchange polymer, an ion exchange polymer metal composite, an ionomer base material. In some embodiments, the electroactive polymer is perfluorinated polymer such as polytetrafluoroethylene, polyfluorosulfonic acid, perfluorosulfonate, and polyvinylidene fluoride. Other suitable polymers include polyethylene, polypropylene, polystyrene, polyaniline, polyacrylonitrile, cellophane, cellulose, regenerated cellulose, cellulose acetate, polysulfone, polyurethane, polyvinyl alcohol, polyvinyl acetate, polyvinyl pyrrolidone. Typically, the electroactive polymer element includes a biocompatible conductive material such as platinum, gold, silver, palladium, copper, and/or carbon.

Suitable shapes of the electroactive polymer element include three dimensional shape, substantially rectangular, substantially triangular, substantially round, substantially trapezoidal, a flat strip, a rod, a cylindrical tube, an arch with uniform thickness or varying thickness, a shape with slots that are perpendicular to the axis, slots that are parallel to the longitudinal axis, a coil, perforations, and/or slots.

IPMC is a polymer and metal composite that uses an ionomer as the base material. Ionomers are types of polymers that allow for ion movement through the membrane. There are several ionomers available in the market and some of the suited ionomers for this application are polyethylene, polystyrene, polytetrafluoroethylene, polyvinylidene fluoride, polyfluorosulfonic acid based membranes like NAFION® (from E. I. Du Pont de Nemours and Company, Wilmington, Del.), polyaniline, polyacrylonitrile, cellulose, cellulose acetates, regenerated cellulose, polysulfone, polyurethane, or combinations thereof. A conductive metal, for example gold, silver, platinum, palladium, copper, carbon, or combinations thereof, can be deposited on the ionomer to make the IPMC. The IPMC element can be formed into many shapes, for example, a strip, rod, cylindrical tube, rectangular piece, triangular piece, trapezoidal shape, arch shapes, coil shapes, or combinations thereof. The IPMC element can have perforations or slots cut in them to allow tissue in growth.

The electroactive polymer element has, in some embodiments, multiple layers of the electroactive polymer with or without an insulation layer separating the layers of the electroactive polymer. Suitable insulation layers include, but are not limited to, silicone, polyurethane, polyimide, nylon, polyester, polymethylmethacrylate, polyethylmethacrylate, neoprene, styrene butadiene styrene, or polyvinyl acetate.

In some embodiments, the actuator element, the entire device, or portions of the airway implant have a coating. The coating isolates the coated device from the body fluids and/or tissue either physically or electrically. The device can be coated to minimize tissue growth or promote tissue growth. Suitable coatings include poly-L-lysine, poly-D-lysine, polyethylene glycol, polypropylene, polyvinyl alcohol, polyvinylidene fluoride, polyvinyl acetate, hyaluronic acid, and/or methylmethacrylate.

### EMBODIMENTS OF THE DEVICE

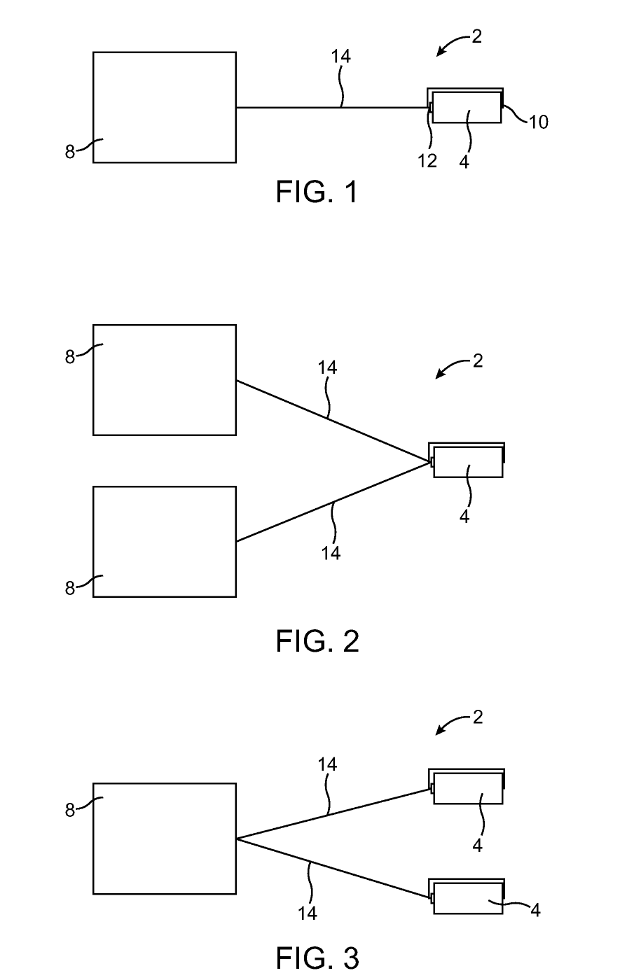

FIG. 1 illustrates an airway implant system 2 that has a power source 4, a connecting element, such as a wire lead 14, and an actuator element, such as an electroactive polymer element 8. Suitable power sources 4 are a power cell, a battery, a capacitor, a substantially infinite bus (e.g., a wall outlet leading to a power generator), a generator (e.g., a portable generator, a solar generator, an internal combustion generator), or combinations thereof. The power source 4 typically has a power output of from about 11 mA to about 5A, for example about 500 mA.

Instead of or in addition to wire lead 14, the connecting element may be an inductive energy transfer system, a conductive energy transfer system, a chemical energy transfer system, an acoustic or otherwise vibratory energy transfer system, a nerve or nerve pathway, other biological tissue, or combinations thereof. The connecting element is made from one or more conductive materials, such as copper. The connecting element is completely or partially insulated and/or protected by an insulator, for example polytetrafluoroethylene (PTFE). The insulator can be biocompatible. The power source 4 is typically in electrical communication with the actuator element 8 through the connecting element. The connecting element is attached to an anode 10 and a cathode 12 on the power source 4. The connecting elements can be made from one or more sub-elements.

The actuator element 8 is preferably made from an electroactive polymer. Most preferably, the electroactive polymer is an ion exchange polymer metal composite (IPMC). The IPMC has a base polymer embedded, or otherwise appropriately mixed, with a metal. The IPMC base polymer is preferably perfluoronated polymer, polytetrafluoroethylene, polyfluorosulfonic acid, perfluorosulfonate, polyvinylidene fluoride, hydrophilic polyvinylidene fluoride, polyethylene, polypropylene, polystyrene, polyaniline, polyacrylonitrile, cellophane, cellulose, regenerated cellulose, cellulose acetate, polysulfone, polyurethane, polyvinyl alcohol, polyvinyl acetate and polyvinyl pyrrolidone, or combinations thereof. The IPMC metal can be platinum, gold, silver, palladium, copper, carbon, or combinations thereof.

FIG. 2 illustrates that the actuator element 8 can have multiple elements 8 and connecting elements 14 that all connect to a single power source 4.

FIG. 3 illustrates an airway implant system 2 with multiple power sources 4 and connecting elements 14 that all connect to a single actuator element 8. The airway implant system 2 can have any number and combination of actuator elements 8 connected to power sources 4.

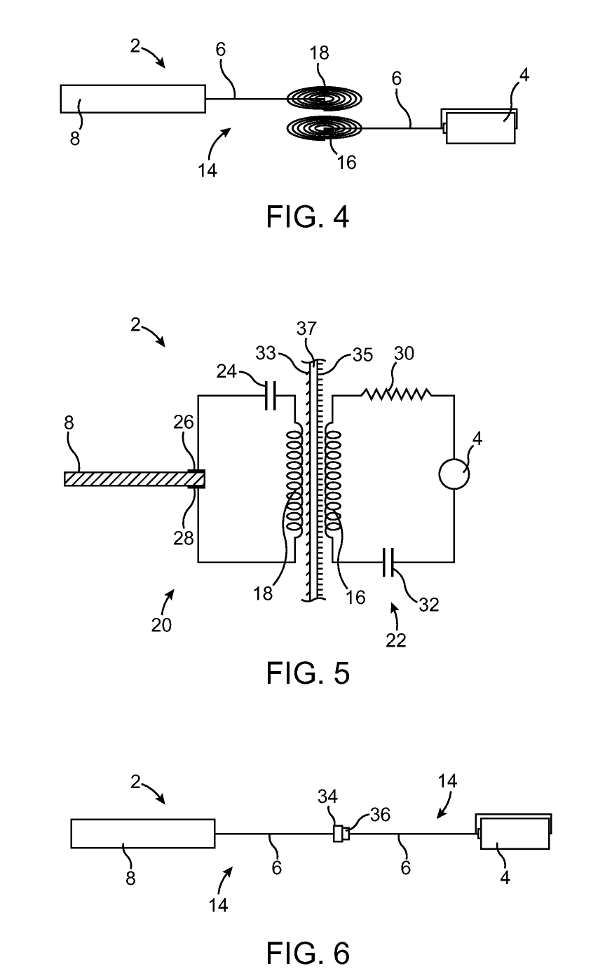

FIG. 4 illustrates an embodiment with the connecting element having a first energy transfer element, for example a first transducer such as a first receiver, and a second energy transfer element, for example a second transducer such as a second inductor 16. In this embodiment, the first receiver is a first inductor 18. The first inductor 18 is typically positioned close enough to the second inductor 16 to enable sufficient inductive electricity transfer between the second and first inductors 16 and 18 to energize the actuator element 8. The connecting element 14 has multiple connecting elements 6.

FIG. 5 illustrates that the airway implant device of the present invention can have an implanted portion 20 and a non-implanted portion 22. In this embodiment, the implanted portion 20 is a closed circuit with the first inductor 18 in series with a first capacitor 24 and the actuator element 8. The actuator element 8 is attached to the closed circuit of the implanted portion 20 by a first contact 26 and a second contact 28. In some embodiments, the implanted portion has a resistor (not shown). The non-implanted portion 22 is a closed circuit. The non-implanted portion 22 has a second inductor 16 that is in series with a resistor 30, the power source 4, and a second capacitor 32. The capacitors, resistors, and, in-part, the inductors are representative of the electrical characteristics of the wire of the circuit and not necessarily representative of specific elements. The implanted portion 20 is within tissue and has a tissue surface 33 nearby. The non-implanted portion is in insulation material 35. An air interface 37 is between the tissue surface 33 and the insulation material 35.

FIG. 6 illustrates an embodiment in which the first energy transfer element of the connecting element 14 is a first conductor 34. The second energy transfer element of the connecting element 14 is a second conductor 36. The first conductor 34 is configured to plug into, receive, or otherwise make secure electrical conductive contact with the second conductor 36. The first conductor 34 and/or second conductor 36 are plugs, sockets, conductive dental fillings, tooth caps, fake teeth, or any combination thereof.

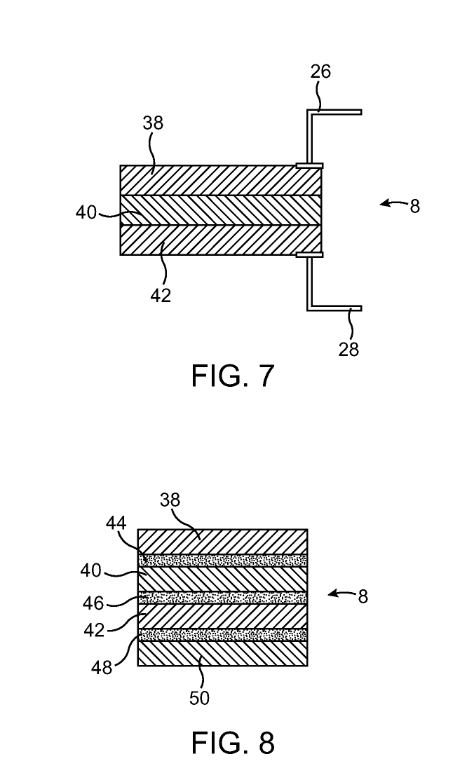

FIG. 7 illustrates an embodiment in which the actuator element 8 is a multi-layered device. The actuator element 8 has a first EAP layer 38, a second EAP layer 40, and a third EAP layer 42. The EAP layers 38, 40 and 42 are in contact with each other and not separated by an insulator.

FIG. 8 illustrates another embodiment in which the actuator element 8 has a first EAP layer 38 separated from a second EAP layer 40 by a first insulation layer 44. A second insulation layer 46 separates the second EAP layer from the third EAP layer 42. A third insulation layer 48 separates the third EAP layer from the fourth EAP layer 50. Insulation material is preferably a polymeric material that electrically isolates each layer. The insulation can be, for example, acrylic polymers, polyimide, polypropylene, polyethylene, silicones, nylons, polyesters, polyurethanes, or combinations thereof. Each EAP layer, 38, 40, 42 and 50 can be connected to a lead wire (not shown). All anodes and all cathodes are connected to the power source 4.

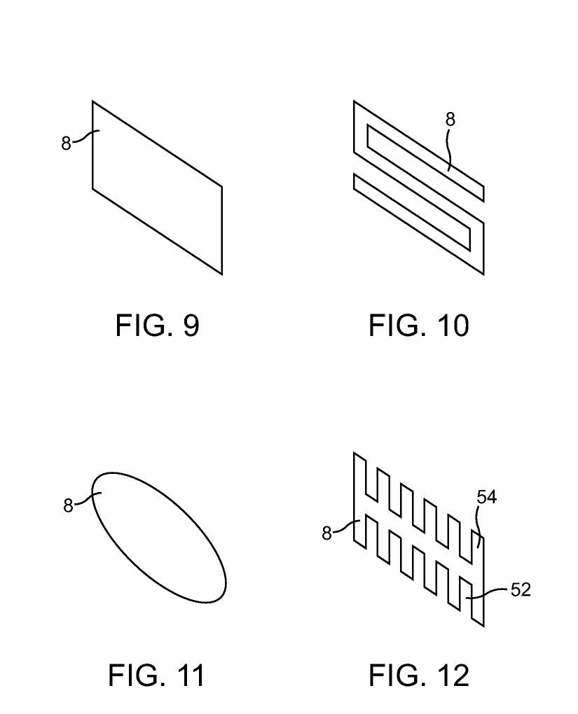

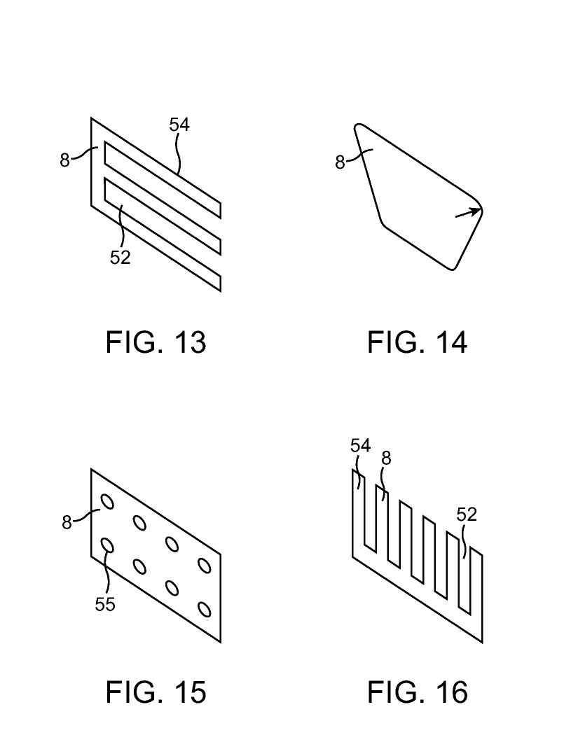

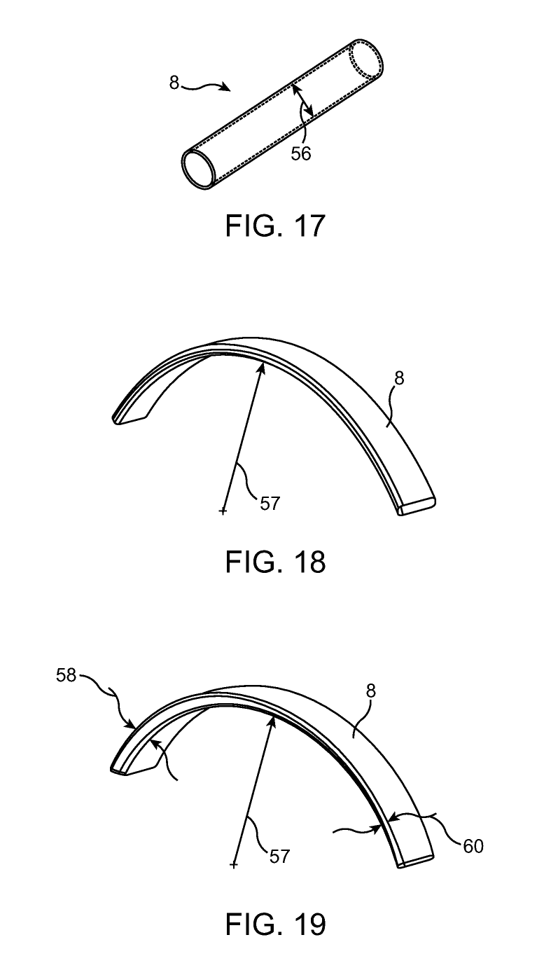

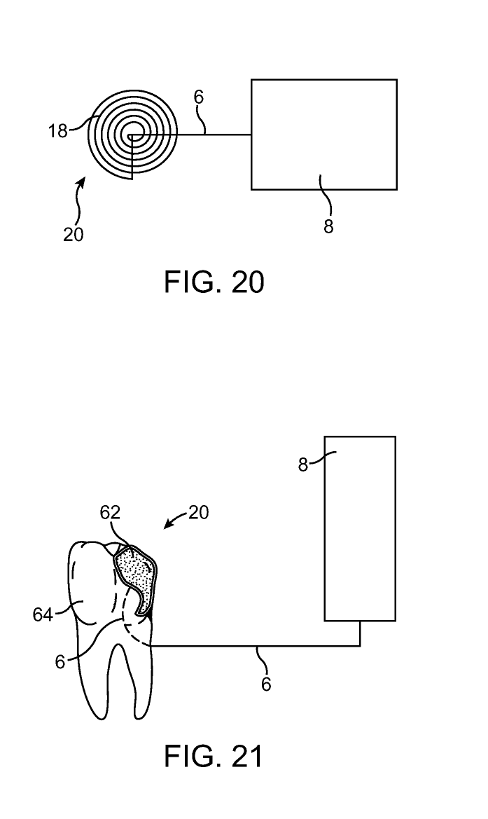

FIGS. 9-19 illustrate different suitable shapes for the actuator element 8. FIG. 9 illustrates a actuator element 8 with a substantially flat rectangular configuration. The actuator element 8 can have a width from about 2 mm to about 5 cm, for example about 1 cm. FIG. 10 illustrates an actuator element 8 with an “S” or zig-zag shape. FIG. 11 illustrates the actuator element 8 with an oval shape. FIG. 12 illustrates a actuator element 8 with a substantially flat rectangular shape with slots 52 cut perpendicular to the longitudinal axis of the actuator element 8. The slots 52 originate near the longitudinal axis of the actuator element 8. The actuator element 8 has legs 54 extending away from the longitudinal axis. FIG. 13 illustrates an actuator element 8 with slots 52 and legs 54 parallel with the longitudinal axis. FIG. 14 illustrates an actuator element be configured as a quadrilateral, such as a trapezoid. The actuator element 8 has chamfered corners, as shown by radius. FIG. 15 illustrates an actuator element 8 with apertures 55, holes, perforations, or combinations thereof. FIG. 16 illustrates an actuator element 8 with slots 52 and legs 54 extending from a side of the actuator element 8 parallel with the longitudinal axis. FIG. 17 illustrates an actuator element 8 with a hollow cylinder, tube, or rod. The actuator element has an inner diameter 56. FIG. 18 illustrates an arched actuator element 8. The arch has a radius of curvature 57 from about 1 cm to about 10 cm, for example about 4 cm. The actuator element 8 has a uniform thickness. FIG. 19 illustrates an arched actuator element 8. The actuator element 8 can have a varying thickness. A first thickness 58 is equal or greater than a second thickness 60.

FIG. 20 illustrates an embodiment of the implanted portion of an airway implant with a coil-type inductor 18 connected by a wire lead 6 to the actuator element 8. In another embodiment, as illustrated in FIG. 21 the implanted portion has a conductive dental filling 62 in a tooth 64. The dental filling 62 is previously implanted for reasons related or unrelated to using of the airway implant system. The dental filling 62 is electrically connected to the wire lead 6. For example, a portion of the wire lead 6 is implanted in the tooth 64, as shown by phantom line. The wire lead 6 is connected to the actuator element 8.

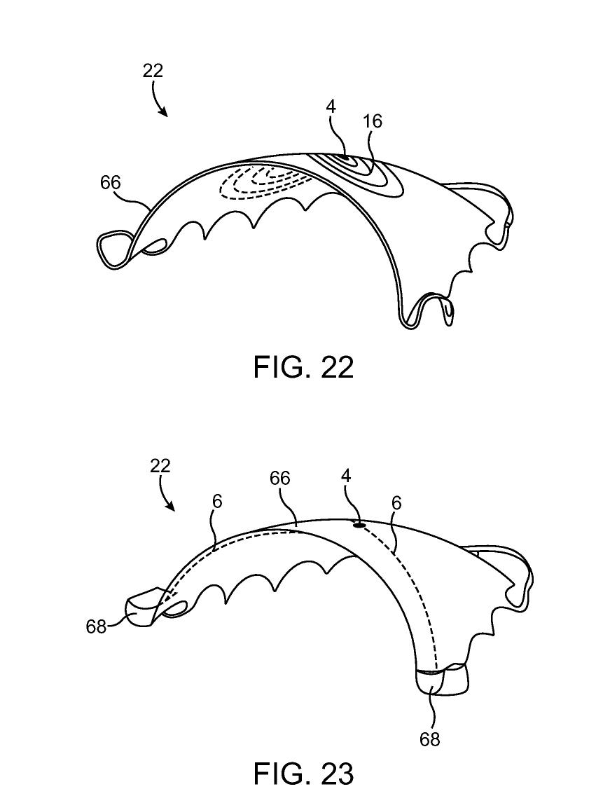

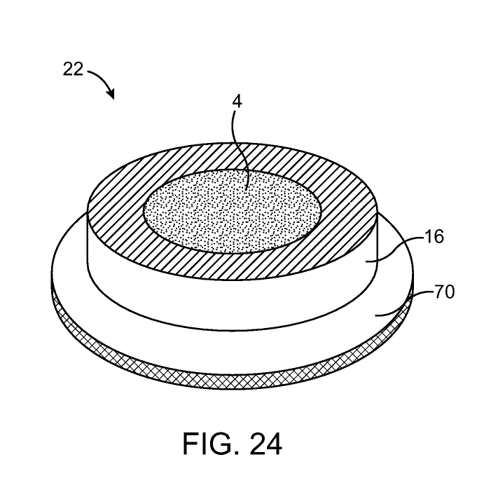

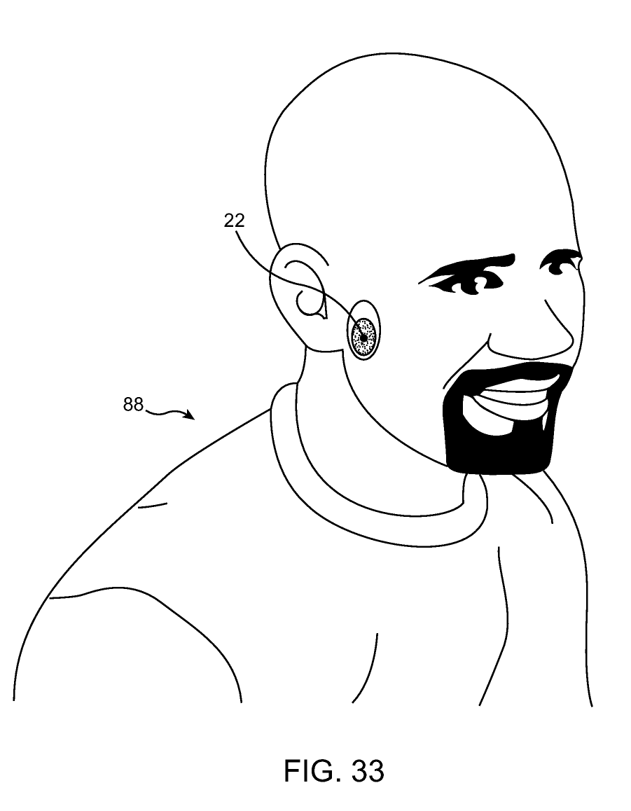

FIG. 22 illustrates an embodiment of the non-implanted portion 22 with a mouthpiece, such as a retainer 66. The retainer 66 is preferably custom configured to fit to the patient's mouth roof, or another part of the patient's mouth. The second transducer, such as second inductor 16, is integral with, or attached to, the retainer 66. The second inductor 16 is located in the retainer 66 so that during use the second inductor 16 is proximal with the first inductor 18. The power source 4, such as a cell, is integral with, or attached to, the retainer 66. The power source 4 is in electrical communication with the second inductor 16. In some embodiments, the retainer 66 has a pulse-width-modulation circuit. FIG. 23 illustrates that the retainer 66 has one or more tooth sockets 68. The tooth sockets 68 are preferably configured to receive teeth that have dental fillings. The tooth sockets 68 are electrically conductive in areas where they align with dental fillings when in use. The power source 4 is connected with the tooth sockets 68 via the wire leads 6. In the embodiment of FIG. 24, the non-implantable portion 22 has the second inductor 16 attached to a removably attachable patch 70. The patch 70 is attached to the power source 4. The power source 4 is in contact with the second inductor 16. This embodiment can be, for example, located on the cheeks as shown on FIG. 33 or any other suitable location.

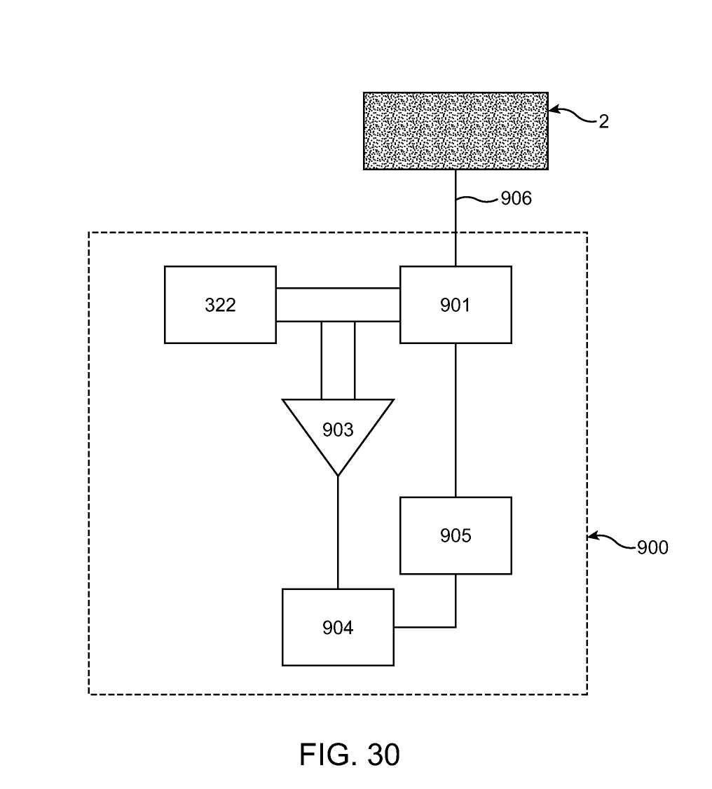

Preferably, the airway implant device 2 discussed herein is used in combination with an inductive coupling system 900 such as depicted in FIG. 30. FIG. 30 depicts an inductive coupling system that is suitable for controlling the airway implant device 2 which includes a connecting element 906 (which connects the electrical contacts (not shown) to the rest of the electrical system), a connector 901, a energy source 322, a sensor 903, a timer 904, and a controller 905. The connector 901, energy source 322, sensor 903, a timer 904, and controller 905 are located in a housing disposed in a region outside or inside the body.

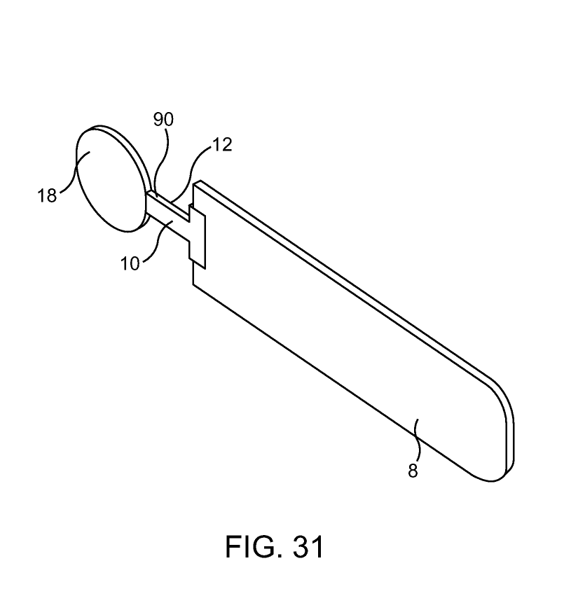

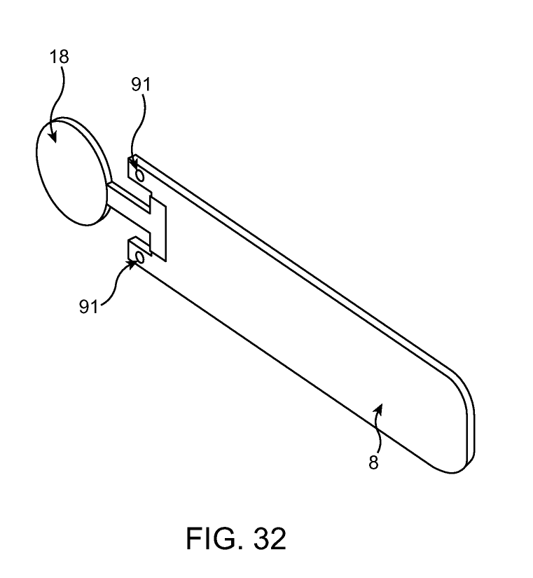

Two preferred embodiments of the airway implant device are shown in FIGS. 31 and 32. The device in FIG. 31 includes the actuator element 8 connected to an anode 10 and cathode 12 and to the induction coil 18. The device also includes a controller 90, such as a microprocessor. The circuitry within the controller is not shown. The controller 90 picks up AC signals from the induction coil 18 and converts it to DC current. The controller 90 can also include a time delay circuit and/or a sensor. The sensor could sense the collapsing and/or narrowing of the airways and cause the device to energize the actuator element 8 and thus completely or partially open up the airway in which the device is implanted. FIG. 32 shows an embodiment with anchors 91 located on the actuator element 8. The implant can be anchored in a suitable location with the use of these anchors and sutures and/or surgical glue.

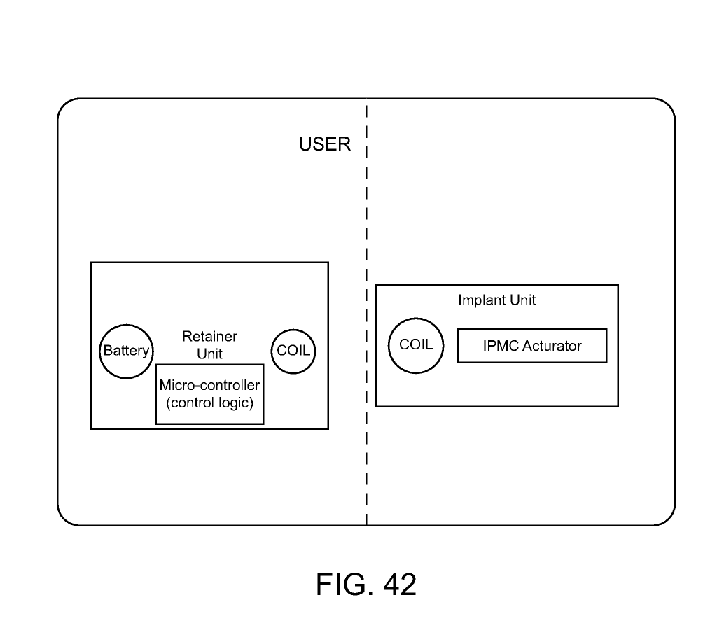

FIG. 42 depicts an embodiment of the invention. The airway implant device comprises of two units—an implant unit and a retainer unit. The implant unit is implanted in a patient and includes an IPMC actuator and a coil. The retainer unit is typically not implanted in the patient and can be worn by the patient prior to going to bed. This unit includes a coil, a battery, and a microcontroller.

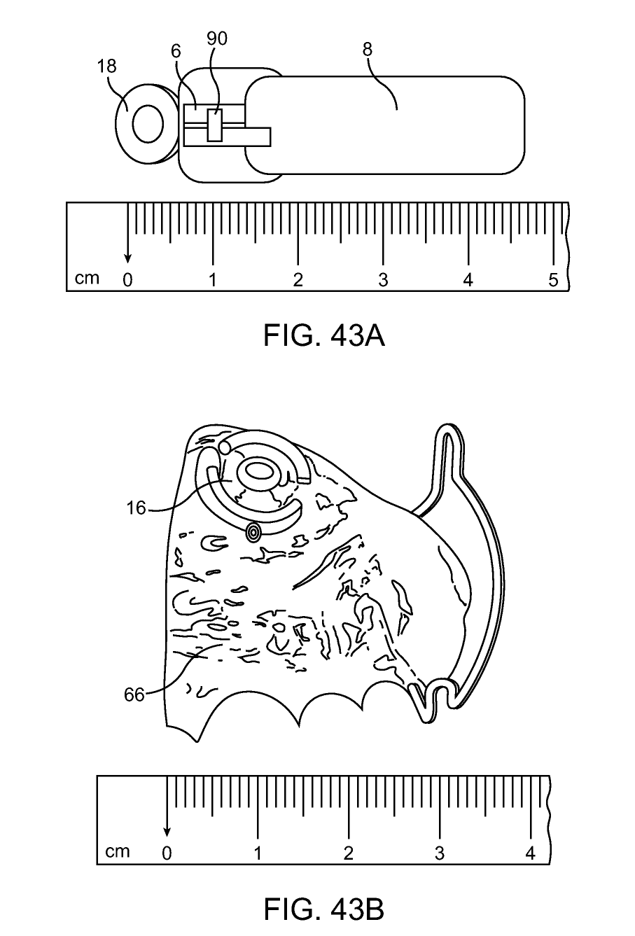

FIG. 43 depicts yet another embodiment of the invention. FIG. 43A is the implant unit, preferably for implantation proximal to or in an airway wall. The implant unit includes an actuator element 8, an inductor 18 in the form of a coil, a controller 90, and connecting elements 6. FIG. 43B depicts the removable retainer with an inductor 16 and a retainer 66.

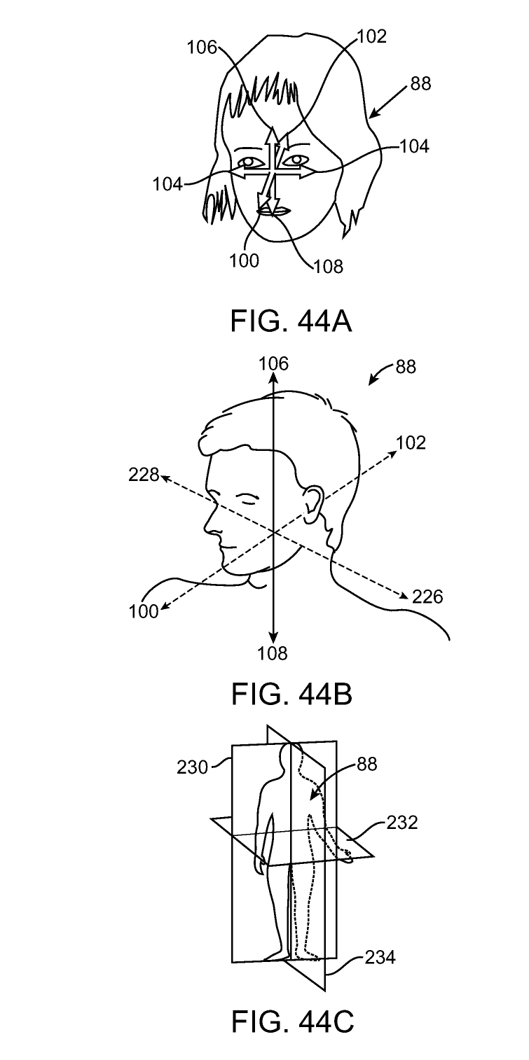

FIGS. 44A, 44B, and 44C illustrate terms used in describing the anatomy of a patient 88 and orientation attributes of the invention. Anterior 100 refers to a part of the body or invention toward the front of the body or invention, or in front of another part of the body or invention. Posterior 102 refers to a part of the invention or body toward the back of the invention or body, or behind another part of the invention or body. Lateral 104 refers to a part of the invention or body to the side of the invention or body, or away from the middle of the invention or body or the middle of the invention or body. Superior 106 refers to a part of the invention or body toward the top of the invention or body. Inferior 108 refers to a part of the invention or body toward the bottom of the invention or body. FIG. 44B illustrates the left 226 and the right 228 sides of a patient anatomy. Various planes of view are illustrated in FIG. 44C, including a coronal plane 230, a transverse plane 232, and a sagittal plane 230.

A preferred embodiment of the device of the present invention comprises an implanted portion 20 comprising an implantable actuator element 8, a housing 112, a first inductor 18, and connecting elements 14 connecting the actuator element 8 to the first inductor 18 within the housing 112; and a non-implanted portion 22 comprising a power source 4 and a second inductor 16 capable of transferring energy to the first inductor 18, wherein the energy of the first inductor 18 energizes the actuator element 8 wherein the actuator element 8 comprises an electroactive polymer element. In a preferred embodiment, the actuator element 8 of the device is implanted in the soft palate 84. The housing 112 of the preferred embodiment is implanted inferior to the hard palate 74. In a preferred embodiment of the device, the housing 112 comprises at least one of acrylic, polytetrafluoroethylene (PTFE), polymethylmethacrylate (PMMA), Acrylonitrile Butadiene Styrene (ABS), polyurethane, polycarbonate, cellulose acetate, nylon, and a thermoplastic or thermosetting material.

In a preferred embodiment, the non-implanted portion 22 is in the form of a mouth guard or dental retainer 66. In a preferred embodiment, the non-implanted portion comprises a non-implantable wearable element. In some embodiments, the superior side of the housing 112 comports to the shape of a hard palate 74. In some embodiments, the housing 112 is cast from an impression of a hard palate 74. In still other embodiments, the housing 112 is concave on its superior side. In some embodiments, the housing 112 is convex on its superior side. In some embodiments, the housing 112 comprises bumps 114 on its superior side lateral to a central axis extending from the housing's 112 anterior to its posterior end. In some embodiments, the housing 112 configuration has a substantially smooth rounded superior side. Other configurations for the housing 112 may be contemplated by one having skill in the art without departing from the invention.

In some embodiments, the actuator element 8 is at least partially within the housing 112. In other embodiments, the actuator element 8 is outside the housing 112. The housing 112 is capable of housing and protecting the first inductor 18 and connecting elements 14 between the first inductor 18 and the actuator element 8. In some embodiments, the housing 112 has a roughened surface to increase friction on the housing 112. In some embodiments, the roughened surface is created during casting of the housing 112. In some embodiments, the roughened surface induces fibrosis.

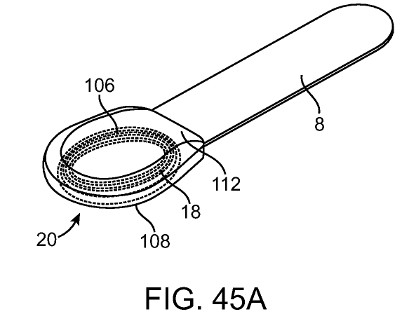

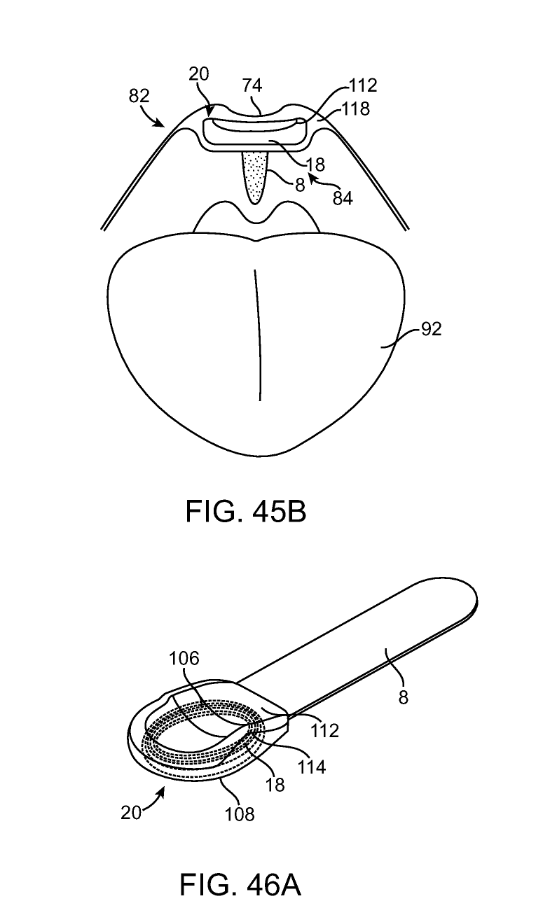

FIG. 45A illustrates one embodiment of the airway implant device comprising a actuator element 8, a first inductor 18, and a housing 112 made from an acrylic and cast with substantially smooth rounded superior and anterior sides. In this embodiment, the actuator element 8 anterior end terminates at about the posterior end of the acrylic housing 112. FIG. 45B illustrates the implant device of FIG. 45A viewed from the anterior side of the implant device, looking toward the posterior end, wherein the implant device is implanted in the palate 116. In the embodiment shown in FIG. 45B, the implant device is implanted such that the housing 112 is in the periosteum 118 inferior to the ridge of the hard palate 74, and the actuator element 8 extends into the soft palate 84.

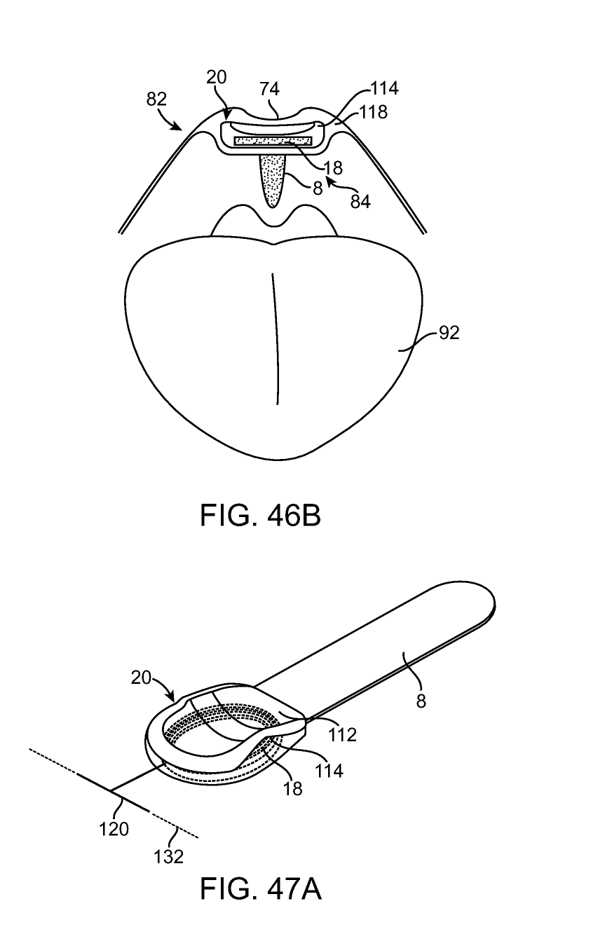

FIG. 46A illustrates an embodiment of the airway implant device that has a actuator element 8, a first inductor 18, and a housing 112 with a smooth rounded inferior side, and at least two bumps 114 on its superior side which, when implanted, comport with the lateral sides of the ridge of the hard palate 74, as shown in FIG. 46B. This configuration reduces rocking of the implant device on the ridge of the hard palate 74 when implanted. In this embodiment, the actuator element 8 anterior end terminates at about the posterior end of the acrylic housing 112. FIG. 46B illustrates the airway implant device of FIG. 46A, viewed from the anterior side of the implant, looking toward the posterior end, wherein the implant device is implanted in the palate 116. In the embodiment shown in FIG. 46B, the implant device is implanted such that the housing 112 is in the periosteum 118 inferior to the ridge of the hard palate 74, and the actuator element 8 extends into the soft palate 84.

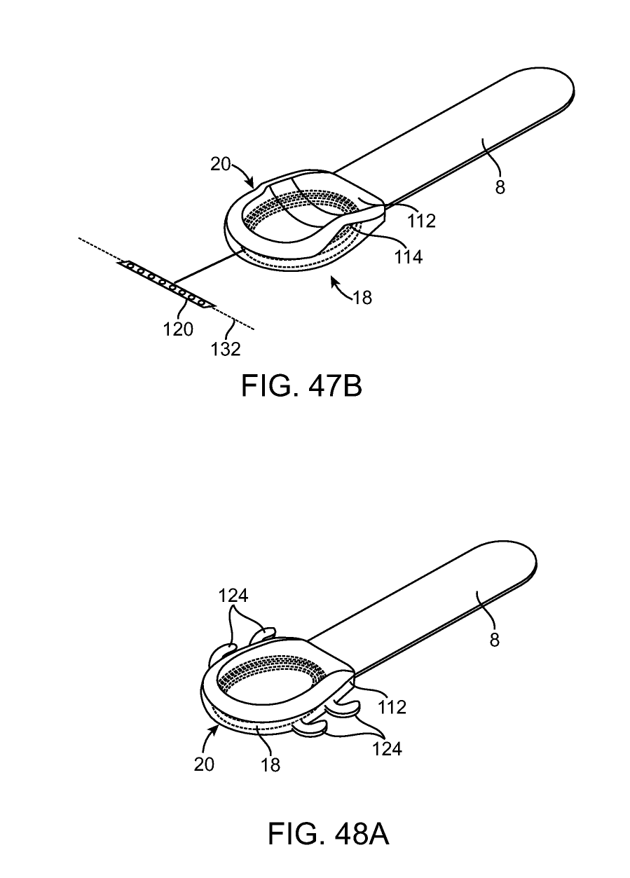

FIG. 47A illustrates an embodiment of the airway implant device having an attachment element 120 at the anterior end of the implant. In this embodiment, the attachment element 120 is T-shaped, however, other configurations and geometries of the attachment element 120 are contemplated in other embodiments, including triangular, circular, L-shaped, Z-shaped, and any geometry within the contemplation of one skilled in the art that would allow attachment of the attachment element to tissue at the anterior end of the implant to fix the position of the implant within the implant cavity.

In some embodiments of the airway implant device having attachment elements 120, the attachment element 120 is a bioabsorbable material. Examples of bioabsorbable materials include, but are not limited to, polylactic acid, polyglycolic acid, poly(dioxanone), Poly(lactide-co-glycolide), polyhydroxybutyrate, polyester, poly(amino acid), poly(trimethylene carbonate) copolymer, poly (ε-caprolactone) homopolymer, poly (ε-caprolactone) copolymer, polyanhydride, polyorthoester, polyphosphazene, and any bioabsorbable polymer.

In another embodiment, the airway implant device comprises an attachment element 120, as shown in FIG. 47B wherein the perforated attachment element 120 comprises at least one hole 122. The hole provides a means for a suture or other attaching device to affix the device to tissue and secure the implant device position. In the case where a suture 132 is used, the suture may or may not be the same suture used by a practitioner to close the original incision made to create a cavity for the implant. The attaching device comprises at least one of a suture, clip, staple, tack, and adhesive.

In some embodiments, the implant may be secured in place, with or without use of an attachment element 120, using an adhesive suitable for tissue, such as cyanoacrylates, and including, but not limited to, 2-octylcyanoacrylate, and N-butyl-2-cyanoacrylate.

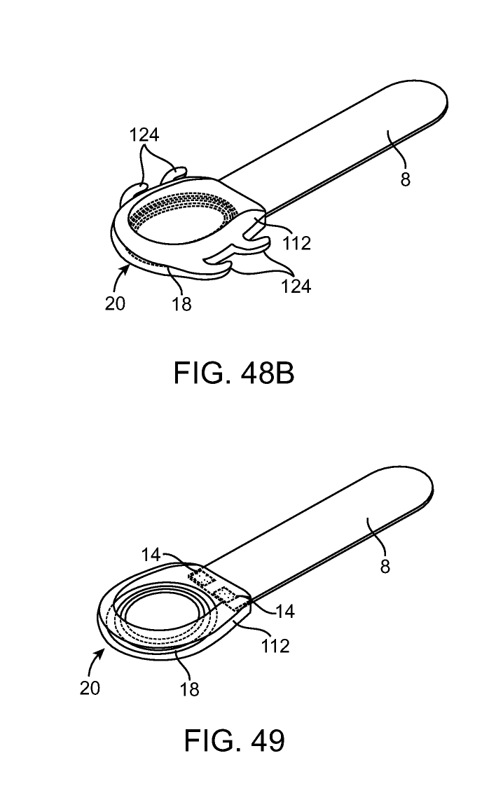

FIG. 48 illustrates an embodiment of the airway implant device wherein the housing 112 has at least one anchor 124. In FIG. 48, the device has four saw-blade like directional anchors 124. The anchors 124 may or may not be made of made of the same materials as the housing 112. Such materials include at least one of acrylic, polytetrafluoroethylene (PTFE), polymethylmethacrylate (PMMA), Acrylonitrile Butadiene Styrene (ABS), polyurethane, polycarbonate, cellulose acetate, nylon, and a thermoplastic material. In some embodiments, the device has at least one anchor 124. In some embodiments, the anchor 124 is configured to allow delivery and removal of the implant device with minimal tissue damage. In some embodiments, the anchor 124 is curved. In some embodiments the superior side(s) of the anchor(s) 124 comport with the hard palate 74 surface. In other embodiments, the superior side(s) of the anchor(s) 124 conform to the configuration of the housing 112, options for which are as described elsewhere in this disclosure.

FIG. 49 illustrates a preferred embodiment of the airway implant device wherein the implanted portion 20 comprises power connecting elements 14 comprising a first contact 26 and a second contact 28. In this embodiment, the first contact 26 and second contact 28 have opposing electrical charges, and the housing 112 encases the contacts. In the embodiment shown, the first contact 26 faces in the inferior direction, while the second contact 28 faces in the superior direction. In other embodiments, the first contact 26 faces in the superior direction while the second contact 28 faces in the inferior direction. In some embodiments, the connecting element 14 comprises a non-corrosive conductive material. In some embodiments, the connecting element 14 comprises platinum, gold, silver, stainless steel, or conductive carbon. In some embodiments, the connecting element 14 comprises stainless steel or copper plated with gold, platinum, or silver. In some embodiments, the actuator element 8 stiffens in one direction when a charge is applied to the connecting element 14. In some embodiments, the actuator element 8 deflects when a charge is applied to the connecting element 14.

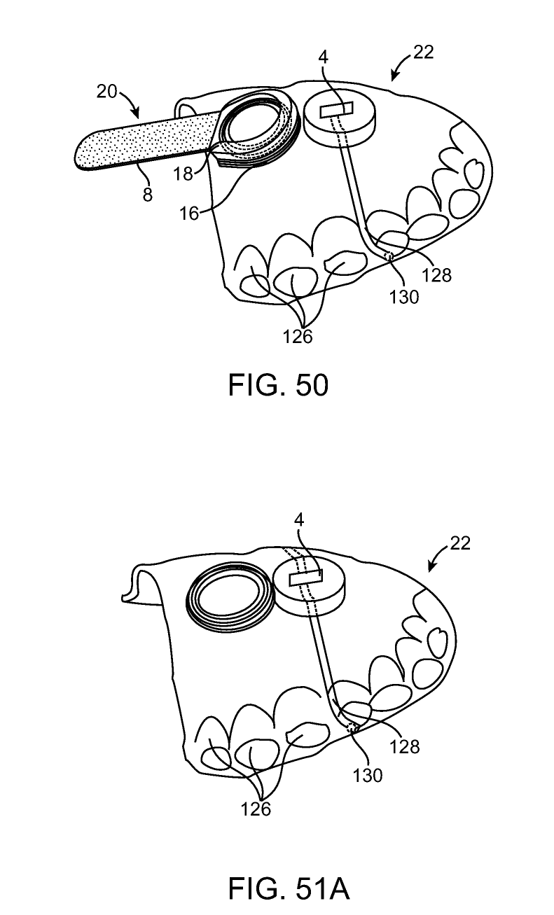

FIG. 50 illustrates an embodiment of the airway implant system wherein the device comprises a non-implanted portion 22 in the form of, and made from similar material as a dental retainer 66. The retainer 66 depicted in FIG. 50 has teeth impressions 126 corresponding to a patient's approximate or exact dentition. Example dental retainer materials include acrylate, polymethylmethacrylate (PMMA), polycarbonate, and nylon. In the embodiment shown in FIG. 50, the non-implanted portion comprises a power source 4 that is rechargeable, a second inductor 16 connected to the power source 4, and ball clamps 128 having two exposed portions 130, said ball clamps 128 connected to the rechargeable power source 4, whereby the exposed portions 130 can recharge the power source 4. The exposed portions 130 are at least partially not covered by retainer material, and are thereby exposed. In the embodiment shown in FIG. 50, the non-implanted portion second inductor 16 transfers energy it receives from the power source 4 to the first inductor 18 of the implanted portion 20, wherein the first inductor 18 energizes the actuator element 8.

In some embodiments, the non-implanted portion 22 does not include ball clamps 128 for recharging the power source 4. In some embodiments, the power source 4 is a rechargeable battery. In some embodiments, the power source 4 is one of a lithium-ion battery, lithium-ion polymer battery, a silver-iodide battery, lead acid battery, a high energy density, or a combination thereof. In some embodiments, the power source 4 is removable from the non-implanted portion 22. In some embodiments, the power source 4 is replaceable. In some embodiments, the power source is designed to be replaced or recharged per a specified time interval. In some embodiments, replacing or recharging the power source 4 is necessary no more frequently than once per year. In other embodiments, replacing or recharging the power source 4 is necessary no more frequently than once every six months. In yet other embodiments, replacing or recharging the power source 4 is necessary no more frequently than once or every three months. In yet another embodiment, daily replacing or recharging of the power source is required.

In some embodiments, the power source 4 and second inductor 16 are sealed within the non-implanted portion and the sealing is liquid proof.

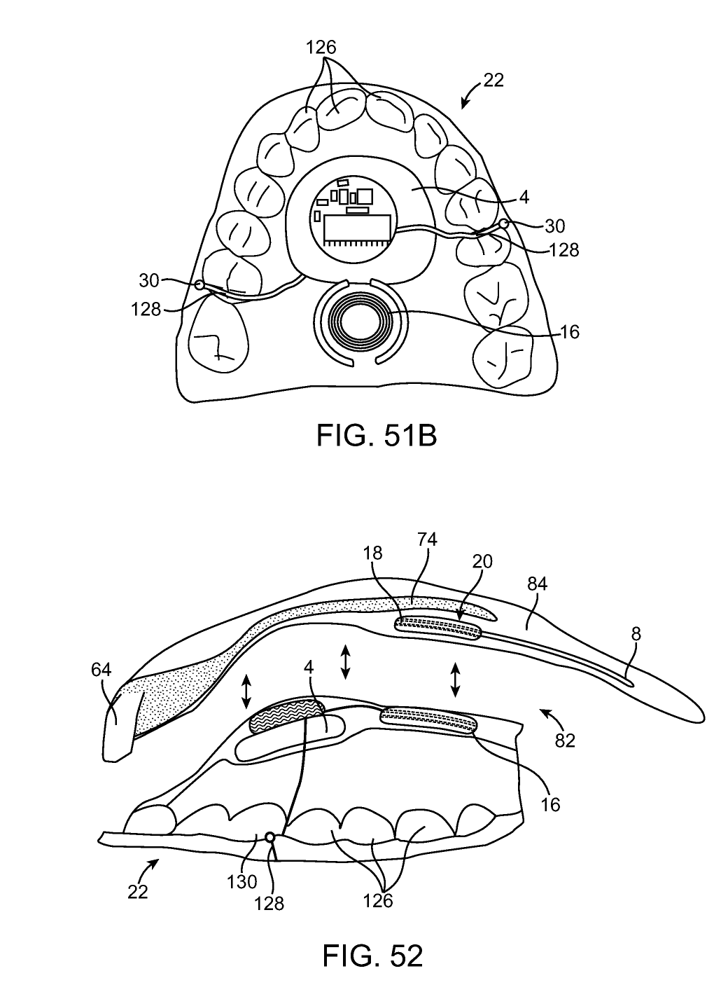

FIGS. 51A, and 51B illustrate different views of an embodiment of the airway implant device non-implanted portion 22 in the form of a retainer 66. In the embodiment depicted, the non-implanted portion 22 comprises a second inductor 16, a power source 4, and at least one ball clamp 128 for recharging the power source 4.

FIG. 52 illustrates an embodiment of the airway implant device implanted in the palate 116. In this embodiment, the housing 112 is implanted inferior to the hard palate 74, whereas the actuator element 8 extends posterior to the housing 112 into the soft palate 84. The non-implanted portion 22 in this embodiment comprises a retainer 66, a power source 4, a second inductor 16, and ball clamps 128 for recharging the power source 4. Other embodiments may comprise none, or some, or all of these elements (the retainer 66, power source 4, second inductor 16, and ball clamps 128), and instead open the airway by means described elsewhere in this specification. In the embodiment depicted in FIG. 52, when the implanted portion 20 of the airway implant device is implanted such that the housing 112 is inferior to the hard palate 74, and when a patient places the retainer 66 in his mouth 82, the retainer 66 having a chargeable second inductor 16 that is positioned within the retainer 66 to align inferior to the implanted first inductor 18, the second inductor 16 transfers energy to the first inductor 18 and the first inductor 18 energizes the actuator element 8. In this embodiment, the actuator element 8 comprises an electroactive polymer (EAP) element, which, when energized by the first inductor 18, opens the airway in which the device is implanted.

The implants described herein are preferably implanted with a deployment tool. Typically, the implantation involves an incision, surgical cavitation, and/or affixing the implant.

### Sensing and Actuation of Airway Implants

One embodiment of the invention is an airway implant device with a sensor for monitoring a condition prior to and/or during the occurrence of an apneic event. Preferably, the sensor monitors for blockage of an airway. The sensor senses the possible occurrence of an apneic event. This sensing of a possible apneic event is typically by sensing a decrease in the airway gap, a change in air pressure in the airway, or a change in air flow in the airway. A progressive decrease in the airway gap triggers the occurrence of an apneic event. Most preferably the sensor senses one or more events prior to the occurrence an apneic event and activates the airway implant to prevent the apneic event. In some embodiments, the airway implant device and the sensor are in the same unit. In other embodiments, the actuator element of the airway implant device is the sensor. In these embodiments, the actuator element acts as both a sensor and actuator. In yet other embodiments, the airway implant device and the sensor are in two or more separate units.

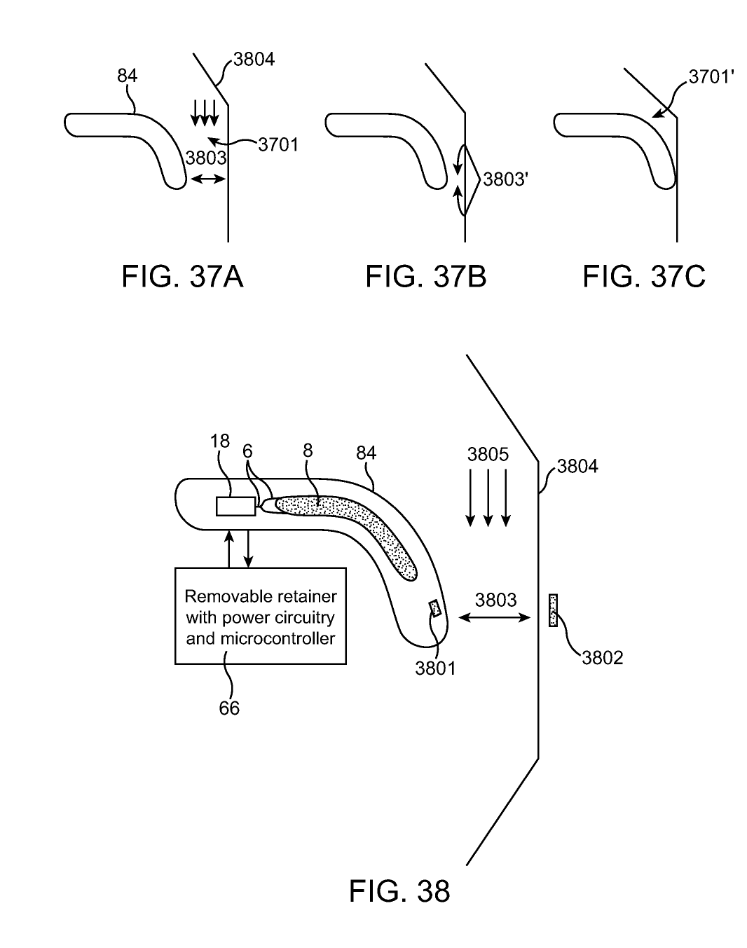

FIG. 37 depicts the occurrence of an apneic event due to the blockage of airway 3701 caused by the movement of the soft palate 84. FIG. 37A shows the soft palate 84 position during normal breathing cycle. An airway gap 3803 is maintained between the soft palate 84 and the laryngeal wall 3804 to maintain airflow 3805. FIG. 37B shows the position of the soft palate 84 just prior to the airway 3701 blockage. It can be seen that the gap 3803′ in this case is smaller than the gap 3803 in FIG. 37A. FIG. 37C shows the soft palate 84 blocking the airway 3701′, leading to the occurrence of an apneic event. In one aspect of the invention, the event shown in FIG. 37C is prevented by taking preemptive action during occurrence of event depicted in FIG. 37B.

One aspect of the invention is an airway implant device with a sensor for sensing the occurrence of apneic events and actuating the device. The invention also includes methods of use of such device.

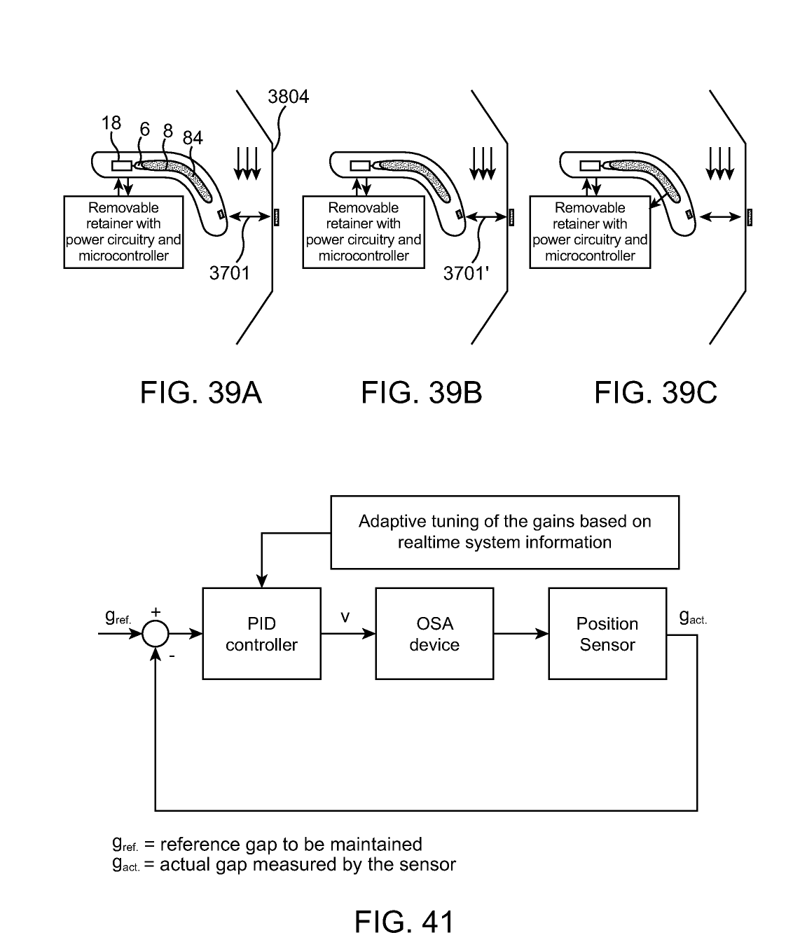

One embodiment of an airway implant device with sensor is depicted in FIG. 38. Non-contact distance sensors 3801 and 3802 are mounted on the laryngeal wall 3804 and also on the soft palate 84 to sense the airway gap between the soft palate 84 and the laryngeal wall 3804. One or more gap values are calibrated into a microcontroller controlling the airway implant device. The functioning of the airway implant device with a sensor is depicted in FIG. 39. During the occurrence of the apneic event the gap between the soft palate 84 and the laryngeal wall 3804 decreases. This gap information is continuously monitored by the airway implant device microcontroller. When the gap becomes smaller than a preset threshold value, the airway implant microcontroller actuates the airway implant, which stiffens the soft palate 84 and the gap between the soft palate 84 and the laryngeal walls 3804 increases. When this gap crosses an upper threshold, the microcontroller powers off the airway implant actuator.

In one embodiment, the operation of the device is as follows:
a) A threshold gap is calibrated into the microcontroller which is present in the removable retainer of the device. This threshold gap corresponds to the gap 3803′ formed by the position of the soft palate with respect to the laryngeal wall as depicted in the FIG. 37B, i.e., a distance at which an apneic event could be triggered or an apneic event occurs. This calibration can take place in real time or when the device is being installed.
b) The non-contact sensor constantly monitors the gap and the information is constantly analyzed by a program present in the microcontroller.
c) The airway implant actuator is in the off state (not powered state) as long as the threshold gap is not reached.
d) When the gap is equal to the threshold gap, the micro controller, powers on the airway implant actuator (on state). This leads to the stiffening of the airway implant actuator, which in-turn stiffens the soft palate.
e) This stiffening of the soft palate prevents the obstruction of the airway and modulates the occurrence of an apneic event.
f) When the gap becomes more than the threshold gap, the micro-controller turns off the airway implant actuator (off state).

Typically, an algorithm in the micro-controller controls the actuation of the actuator. An example of the algorithm is—
if (gap<threshold gap); {Voltage applied to airway implant actuator=high (on state)} or else {Voltage applied to the airway implant actuator=low (off state)}

Complex algorithms, such as adaptive algorithms, can also be used. The objective of the adaptive algorithm can be to selectively control the stiffness of the soft palate by varying the power applied to the airway implant actuator.

Another example of an algorithm to selectively control the stiffness of the soft palate is:

If (gap < or = g)
{Apply full power to the airway implant actuator}
Else
If (gap = g1)
{Voltage applied to airway implant actuator = v1}
Else if (gap = g2)
{Voltage applied to airway implant actuator = v2}
Else if (gap = g3)
{Voltage applied to airway implant actuator = v3}
Note (g1, g2, g3 > g)

An example of a controller to maintain a predetermined reference gap is shown is FIG. 41. The objective of this algorithm is to maintain an actual airway gap g_{act} as close to the reference airway gap g_{ref} as possible by controlling the airway implant device actuator. The actual airway gap between the soft palate and the laryngeal wall g_{act} is measured and this information is the output of the position sensor. This airway gap information is feedback to the microcontroller which has a controller algorithm embedded in it. In the microcontroller the g_{act} is compared to a g_{ref} and based on the difference between both, the Proportional Integral Derivative (PID) controller generates a controlling voltage which is supplied to the airway implant device. The PID controller can have fixed gains or can have the gains adaptively tuned based on system information.

In alternative embodiments, the sensor can be a wall tension sensor, an air pressure sensor, or an air flow monitoring sensor. In another embodiment, instead of fully turning the airway implant actuator on or off, the actual value of the airway gap can be used to selectively apply varying voltage to the airway implant actuator, hence selectively varying the stiffness of the soft palate. In yet another embodiment, if the airway implant actuator exhibits a lack of force retention over an extended period of time under DC voltage, a feedback control algorithm may be implemented in the microcontroller, which uses the sensory information provided by the sensors to control the stiffness of the soft palate by maintaining the force developed by the airway implant actuator.

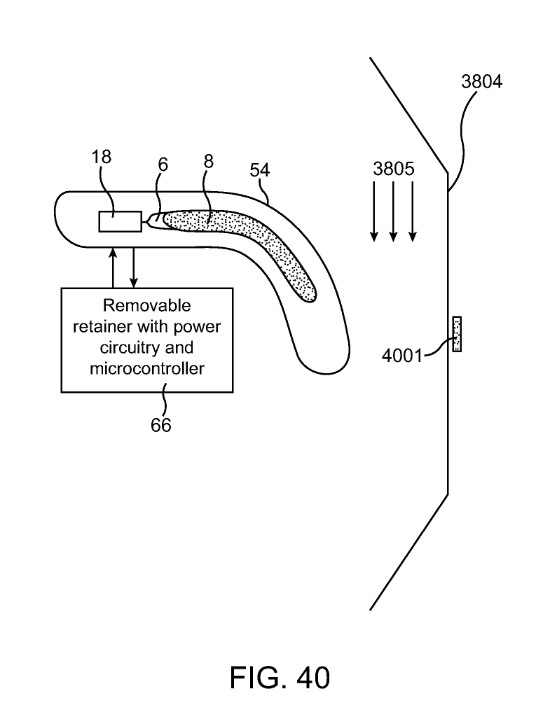

Another embodiment of the invention is depicted in FIG. 40. In this embodiment, the wall tension sensed by the wall tension sensor 4001 implanted into the laryngeal wall 3804 is used as a threshold criterion for activating the airway implant actuator. A wall tension sensor can also be placed in a pharyngeal wall or other suitable airway wall. The sensors of this invention can be placed in an airway wall or proximal to an airway wall.

Some of the advantages of the use of an airway sensor with an airway implant device include: optimization of the power consumed by the airway implant device and hence extension of the life of the device; assistance in predicting the occurrence of apneic event, and hence selective activation of the device in order to minimize any patient discomfort; flexibility to use a feedback control system if required to compensate for any actuator irregularities; and possible configuration of the system to interact with an online data management system which will store different parameters related to apneic events for a patient. This system can be accessed by the doctor, other health care providers, and the insurance agency which will help them provide better diagnosis and understanding of the patient's condition.

In preferred embodiments, the airway gap is individually calculated and calibrated for each patient. This information can be stored in the microcontroller. The sensors are described herein mainly in the context of airway implant devices comprising of electroactive polymer actuators. The sensors can also be used with airway implant devices comprising other active actuators, i.e., actuators that can be turned on, off, or otherwise be controlled, such as magnets. The sensors can be used to activate, in-activate, and/or modulate magnets used in airway implant devices. Preferably, the sensors are in the form of a strip, but can be any other suitable shape for implantation. They are typically deployed with a needle with the help of a syringe. The sensor can be made with any suitable material. In preferred embodiments, the sensor is a smart material, such as an IPMC. The sensor is typically in connection with a microcontroller, which is preferably located in the retainer. This connection can be either physical or wireless.

Suitable sensors include, but are not limited to, an electroactive polymer like ionic polymer metal composite (IPMC). Suitable materials for IPMC include perfluorinated polymer such as polytetrafluoroethylene, polyfluorosulfonic acid, perfluorosulfonate, and polyvinylidene fluoride. Other suitable polymers include polyethylene, polypropylene, polystyrene, polyaniline, polyacrylonitrile, cellophane, cellulose, regenerated cellulose, cellulose acetate, polysulfone, polyurethane, polyvinyl acetate. Typically, the electroactive polymer element includes a biocompatible conductive material such as platinum, gold, silver, palladium, copper, and/or carbon. Commercially available materials suitable for use as a sensor include Nafion® (made by DuPont), Flemion® (made by Asahi Glass), Neosepta® (made by Astom Corporation), Ionac® (made by Sybron Chemicals Inc), Excellion™ (made by Electropure). Other materials suitable for use as a sensor include materials with piezoelectric properties like piezoceramics, electrostrictive polymers, conducting polymers, materials which change their resistance in response to applied strain or force (strain gauges) and elastomers.

The airway implant devices of the present invention, with or without the sensor, can be used to treat snoring. For snoring, the sensor can be adapted and configured to monitor air passageways so as to detect the possible occurrence of snoring or to detect the possible worsening of ongoing snoring. Preferably the sensors are capable of detecting relaxation of tissues in the throat, which can cause them to vibrate and obstruct the airway. Other tissues that can be monitored by the sensor include the mouth, the soft palate, the uvula, tonsils, and the tongue.

Another disease that can be treated with the devices of the present invention includes apnea. The sensor preferably monitors the throat tissue for sagging and/or relaxation to prevent the occurrence of an apneic event. Other tissues that can be monitored by the sensor include the mouth, the soft palate, the uvula, tonsils, and the tongue.

### Methods of Making Electroactive Polymer Element

In some embodiments, the EAP element is an IPMC strip which is made from a base material of an ionomer sheet, film or membrane. The ionomer sheet is formed using ionomer dispersion.

IPMC is made from the base ionomer of for example, polyethylene, polystyrene, polytetrafluoroethylene, polyvinylidene fluoride (PVDF) (e.g., KYNAR® and KYNAR Flex®, from ATOFINA, Paris, France, and SOLEF®, from Solvay Solexis S.A., Brussels, Belgium), hydrophilic-PVDF (h-PVDF), polyfluorosulfonic acid based membranes like NAFION® (from E.I. Du Point de Nemours and Company, Wilmington, Del.), polyaniline, polyacrylonitrile, cellulose, cellulose acetates, regenerated cellulose, polysulfone, polyurethane, and combinations thereof. The conductive material that is deposited on the ionomer can be gold, platinum, silver, palladium, copper, graphite, conductive carbon, or combinations thereof. Conductive material is deposited on the ionomer either by electrolysis process, vapor deposition, sputtering, electroplating, or combination of processes.

The IPMC is cut into the desired implant shape for the EAP element. The electrical contact (e.g., anode and cathode wires for EAP element) is connected to the IPMC surfaces by, for example, soldering, welding, brazing, potting using conductive adhesives, or combinations thereof. The EAP element is configured, if necessary, into specific curved shapes using mold and heat setting processes.

In some embodiments, the EAP element is insulated with electrical insulation coatings. Also, the EAP element can be insulated with coatings that promote cell growth and minimize fibrosis, stop cell growth, or kill nearby cells. The insulation can be a biocompatible material. The EAP element is coated with polymers such as polypropylene, poly-L-lysine, poly-D-lysine, polyethylene glycol, polyvinyl alcohol, polyvinyl acetate, polymethyl methacrylate, or combinations thereof. The EAP element can also be coated with hyaluronic acid. The coating is applied to the device by standard coating techniques like spraying, electrostatic spraying, brushing, vapor deposition, dipping, etc.

In one example, a perfluorosulfonate ionomer, PVDF or h-PVDF sheet is prepared for manufacturing the EAP element. In an optional step, the sheet is roughened on both sides using, for example, about 320 grit sand paper and then about 600 grit sand paper; then rinsed with deionized water; then submerged in isopropyl alcohol (IPA); subjected to an ultrasonic bath for about 10 minutes; and then the sheet is rinsed with deionized water. The sheet is boiled for about 30 minutes in hydrochloric acid (HCL). The sheet is rinsed and then boiled in deionized water for about 30 minutes. The sheet is then subject to ion-exchange (i.e., absorption). The sheet is submerged into, or otherwise exposed to, a metal salt solution at room temperature for more than about three hours. Examples of the metal salt solution are tetraammineplatinum chloride solution, silver chloride solution, hydrogen tetrachloroaurate, tetraamminepalladium chloride monohydrate or other platinum, gold, silver, carbon, copper, or palladium salts in solution. The metal salt solution typically has a concentration of greater than or equal to about 200 mg/100 ml water. 5% ammonium hydroxide solution is added at a ratio of 2.5 ml/100 ml to the tetraammineplatinum chloride solution to neutralize the solution. The sheet is then rinsed with deionized water. Primary plating is then applied to the sheet. The sheet is submerged in water at about 40° C. 5% solution by weight of sodium borohydride and deionized water is added to the water submerging the sheet at 2 ml/180 ml of water. The solution is stirred for 30 minutes at 40° C. The sodium borohydride solution is then added to the water at 2 ml/180 ml of water and the solution is stirred for 30 minutes at 40° C. This sodium borohydride adding and solution stirring is performed six times total. The water temperature is then gradually raised to 60° C. 20 ml of the sodium borohydride solution is then added to the water. The solution is stirred for about 90 minutes. The sheet is then rinsed with deionized water, submerged into 0.1 N HCI for an hour, and then rinsed with deionized water.

In some embodiments, the sheet receives second plating. The sheet is submerged or otherwise exposed to a tetraammineplatinum chloride solution at a concentration of about 50 mg/100 ml deionized water. 5% ammonium hydroxide solution is added at a rate of 2 ml/100 ml of tetrammineplatinum chloride solution. 5% by volume solution of hydroxylamine hydrochloride in deionized water is added to the tetraammineplantium chloride solution at a ratio of 0.1 of the volume of the tetraammineplatinum chloride solution. 20% by volume solution of hydrazine monohydrate in deionized water is added to the tetraammineplatinum chloride solution at a ratio of 0.05 of the volume of the tetraammineplantinum chloride solution. The temperature is then set to about 40° C. and the solution is stirred.

A 5% solution of hydroxylamine hydrochloride is then added at a ratio of 2.5 m/100 ml of tetraammineplatinum chloride solution. A 20% solution of hydrazine monohydrate solution is then added at a ratio of 1.25 ml/100 ml tetraammineplatinum chloride solution. The solution is stirred for 30 minutes and the temperature set to 60° C. The above steps in this paragraph can be repeated three additional times. The sheet is then rinsed with deionized water, boiled in HCl for 10 minutes, rinsed with deionized water and dried.

In some embodiments, the polymer base is dissolved in solvents, for example dimethyl acetamide, acetone, methylethyle ketone, toluene, dimethyl carbonate, diethyl carbonate, and combinations thereof. The solvent is then allowed to dry, producing a thin film. While the solution is wet, a low friction, (e.g., glass, Teflon) plate is dipped into the solution and removed. The coating on the plate dries, creating a think film. The plate is repeatedly dipped into the solution to increase the thickness of the film.

Polyvinyl alcohol, polyvinyl pyrrolidone, polyinyl acetate or combinations thereof can be added to a PVDF solution before drying, thus contributing hydrophilic properties to PVDF and can improve ion migration through the polymer film during manufacture. Dye or other color pigments can be added to the polymer solution.

### Implant Tester

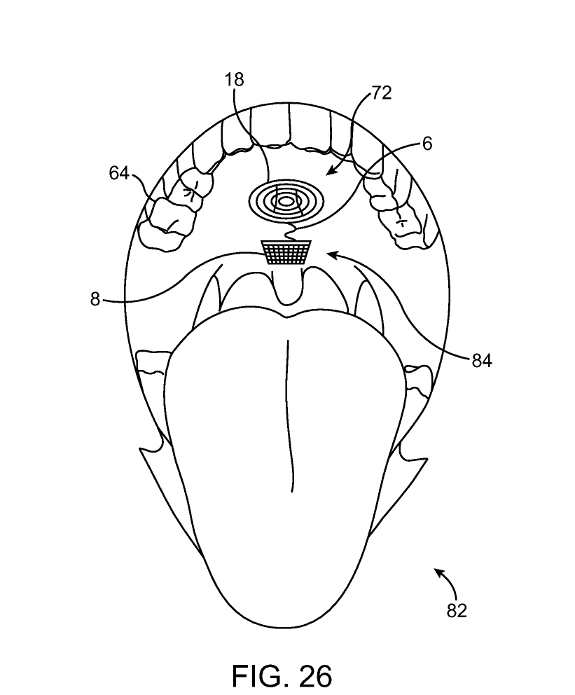

Another aspect of the invention is directed to an implant testing device. From time to time after insertion, it is desirable to verify the presence, location, and proper functioning of the airway implant device. However, because it is inserted subcutaneously in or around a patient's soft palate, access to the device by a technician or treating physician is limited. FIG. 26, for example, shows that elements of the airway implant (including first inductor 18 and actuator 8) are inserted into the roof 72 of the patient's mouth and are therefore not directly accessible after the implant procedure.

With an inductively powered device, it is also useful to test for proper functioning of the pickup coil after it has been implanted in the patient. For example, a continuity test can provide an indication of the coil's ability to receive an inductive power transfer and thus to supply power to the actuator. If the inductor is damaged, it may be incapable of powering the actuator, thereby compromising the implant's ability to control the airway passage. However, as noted above, direct access to the implant device for testing is limited once it has been inserted into the patient.

Finally, following insertion, it may be necessary to accurately determine the position of the implant within the patient. FIG. 52, for example, shows that power transfer electronics of non-implanted portion 22 are positioned relative to power receive electronics of the implant 20. As illustrated, second inductor 16 is aligned with first inductor 18 to ensure effective coupling of the electromagnetic field. Proper alignment of the power transfer electronics presupposes that the location of first inductor 18 within soft palate 84 is known or can be readily ascertained.

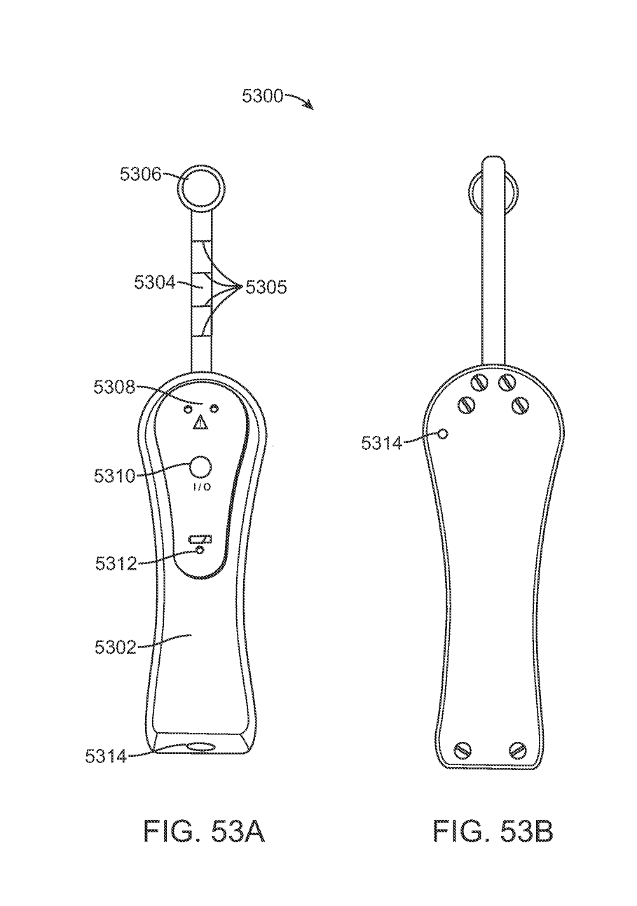

FIGS. 53A-53B illustrate one embodiment of a handheld testing device 5300 according to the present invention. Testing device 5300 can be used to locate an implant within a patient's body and to provide an indication of its power transfer capability. FIG. 53A shows a top surface of testing device 5300 including, in part, handle 5302, elongated portion 5304, and detector 5306. Handle 5302 is adapted for handheld use and, in various embodiments, houses electronics (not shown) used to detect the implant device.

Elongated portion 5304 is connected to handle 5302 and can, for example, be inserted into a patient's mouth for testing a palatal implant. In some embodiments, elongated portion 5304 folds back on handle 5302 and can be secured in a closed position for storage or transport. In other embodiments, elongated portion 5304 retracts into handle 5302 while, in still other embodiments, elongated portion 5304 detaches from handle 5302 and can be removed when not in use.

In some embodiments, elongated portion 5304 includes a positioning scale 5305. When elongated portion 5304 is inserted into a patient's mouth, for example, positioning scale 5305 can indicate a distance from the front teeth to the detector element 5306. The distance can be expressed in centimeters, millimeters, or other convenient units. Using the positioning scale, it is possible to accurately determine a location of the implant device. For example, positioning scale 5305 can be used in the construction or repair of non-implanted portion 22 so as to facilitate alignment of the power transfer electronics in retainer 66 and implant 20. In some embodiments, an angular scale is also included and provides additional positioning information.

Detector 5306 includes an implant detection circuit. In one embodiment, the implant detection circuit emits an electromagnetic field. Metallic objects within a proximity of detector 5306 create disturbances in the electromagnetic field. These disturbances can be detected by a processing circuit. In some embodiments, the processing circuit is disposed within handle 5302, but it can also be located in elongated portion 5304 or externally as required. By monitoring signals from the detection circuit, the processing circuit determines proximity of the implant to detector 5306.

The processing circuit can be configured to detect characteristics associated with the airway implant device and to signal its presence via status indicators 5308. As shown, indicators 5308 can include one or more light emitting diodes (LEDs) or like devices. In one embodiment, indicators 5308 include a green LED which signals proximity of the airway implant and a red LED which indicates an operating status of the testing device 5300. It will be recognized that many variations are possible and within the scope of the present invention. For example, indicators 5308 may signal proximity by changing color or flashing at different rates. Similarly, indicators 5308 may provide audible cues such as tones which change in pitch based on proximity.

Testing device 5300 can also include power-related features such as a battery indicator 5312 and a power/reset switch 5310. In some embodiments, testing device 5300 is powered by, for example, a rechargeable lithium polymer battery. Battery indicator 5312 can include one or more light emitting diodes, a liquid crystal display, or similar elements for providing an indication of battery voltage. Power/reset switch 5310 is used to activate and deactivate testing device 5300 and to perform a reset in the event of a fault or over-modulation condition. Adaptor 5314 permits use of an external power source either as an alternative to battery power or for purposes of recharging the internal battery.

FIG. 53B shows a back surface of testing device 5300. In particular, testing device 5300 includes an additional status indicator 5314 disposed on the back surface. Preferably, status indicator 5314 provides proximity information which is visible to a physician when looking up from below the level of the implant device, thus complementing status indicators 5308. For example, status indicator 5314 may comprise one or more colored LEDs which flash under control of the processing circuit based on the proximity of the implant. In some embodiments, status indicator 5314 includes additional display elements (such as a liquid crystal display).

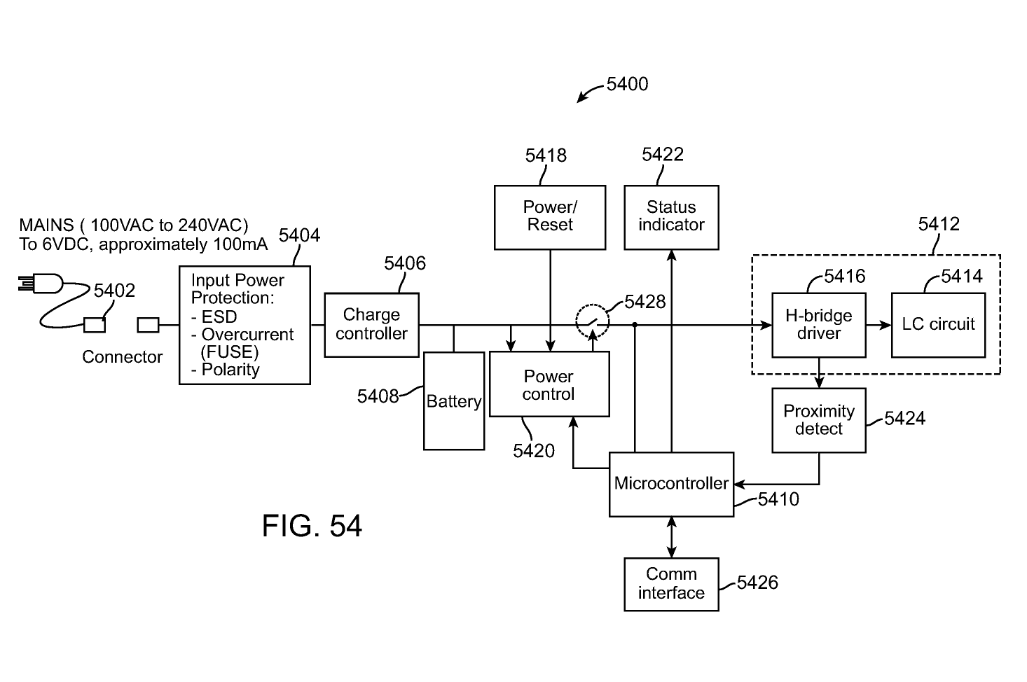

FIG. 54 is a high-level functional block diagram 5400 depicting one embodiment of a testing device according to the present invention. The circuits and electronics described in connection with FIG. 54 can, for example, be disposed in various parts of handheld testing device 5300 including handle 5302, elongated portion 5304, and detector 5306. Alternatively, in some embodiments, the circuits and electronics illustrated in block diagram 5400 can be disposed in an external module which connects to handheld testing device 5300.

Power for operating the testing device can be supplied externally or by a power cell such as a battery. In one embodiment, an external power source is used for charging battery 5408 which, in turn, provides operating power for the testing device. As shown, power connector 5402 is configured to plug into a standard electrical outlet and may include an AC/DC converter for supplying a predetermined voltage and current to the testing device. In other embodiments, power for charging battery 5408 may be supplied wirelessly through an inductive power transfer. When an inductive power transfer is used, the testing device may include a pickup coil and supporting electronics.

Conditioning block 5404 is coupled to power connector 5402 and provides input power protection. For example, conditioning block 5404 may protect device electronics against over-current conditions, electrostatic discharge, and polarity inversions. Charge controller 5406 receives the input voltage from conditioning block 5404 and provides a current for charging battery 5408. Charge controller 5406 also monitors the health of battery 5408 and can halt charging when abnormalities such as high temperature or battery failure are detected.

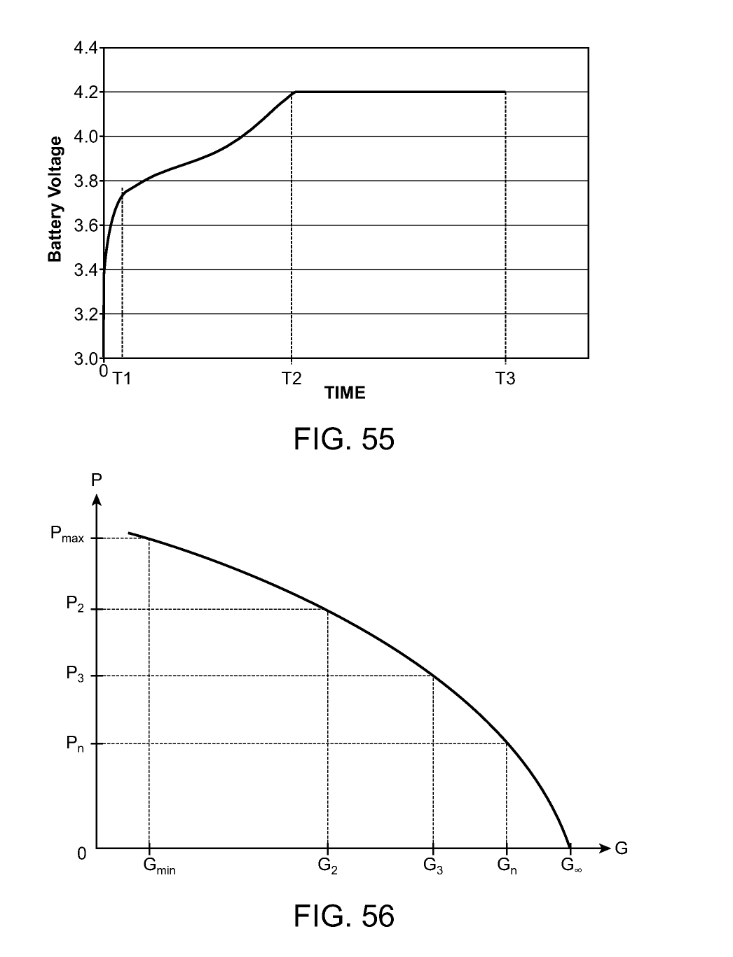

Charge controller 5406 can be configured to provide a constant-current (CC), constant-voltage (CV) charge to battery 5408. Battery 5408, for example, can be a rechargeable lithium polymer cell which supplies a voltage in the range of 3.0-4.2 volts. During a constant-current portion of the charge cycle, current is delivered to battery 5408 at a more or less constant rate thereby increasing the voltage across its terminals. Constant current charging is illustrated in FIG. 55 by the interval from T1 to T2. When the target voltage is reached, charge controller 5406 switches to CV mode and maintains its output at a constant voltage. Constant voltage charging is illustrated in FIG. 55 by the interval from T2 to T3. When low voltage levels are detected at battery 5408, charge controller 5406 can switch to a trickle-charge mode in which charging current delivered to battery 5408 is substantially reduced.

Microcontroller 5410 is configured to control operation of the testing device. Among its many functions, microcontroller 5410 supplies drive signals to a resonator circuit 5412. In one embodiment, the testing device includes an oscillator (not shown) which produces the drive signals. The oscillator may be calibrated before initial use to produce drive signals at the desired frequency. For example, a frequency of the drive signals can be matched to a resonant frequency of the resonator circuit 5412. The drive signals are then applied, under control of the microcontroller 5410, to resonator circuit 5412. Alternatively, the oscillator can be embedded within microcontroller 5410 such that its frequency is adjusted internally by microcontroller 5410.

Resonator circuit 5412 receives drive signals from microcontroller 5410 and produces an electromagnetic field for detecting the implant device. In one embodiment, resonator circuit 5412 includes an LC circuit 5414 and an H-bridge driver 5416. Current flowing through the inductor sets up a magnetic field. As the magnetic field collapses, it charges a capacitor of LC circuit 5414. The capacitor stores the energy in an electric field between its plates. Under the influence of H-bridge drivers 5416, current flows back and forth through the LC circuit 5414 generating an expanding and collapsing electromagnetic field. Although one specific resonator circuit 5412 has been described, many alternatives are possible within the scope of the present invention.

The testing device is activated by power/reset switch 5418. When activated, power control block 5420 closes high-side switch 5428 and allows current to flow from battery 5408 to resonator circuit 5412. Power control block 5420 also deactivates the testing device if an under-voltage condition is detected. For example, microcontroller 5410 can be configured to monitor a voltage level of battery 5408 and to signal power control block 5420 to suspend device operation if the voltage drops below a predetermined cut-off level.

During operation, in one embodiment, microcontroller 5410 drives resonator circuit 5412 at or near its resonant frequency. When operating near the resonant frequency, current flow in resonator circuit 5412 is maximized. A proximity detection circuit 5424 coupled to resonator circuit 5412 monitors resonator current and provides a proximity signal to microcontroller 5410. When the testing device is positioned in the vicinity of a metallic object, a disturbance in the electromagnetic field produced by resonator circuit 5412 is created. For example, as detector 5306 approaches the implant, the implant couples with the electromagnetic field. This coupling alters resonator current flow. Since the resonator circuit 5412 is operating at or near its resonant frequency, such disturbances tend to reduce resonator current flow.

In one embodiment, a proximity detection circuit includes a resistor shunt amplifier and microcontroller 5410 includes an analog-to-digital converter (ADC). Proximity detection circuit 5424 communicates changes in resonator current 5412 as an analog voltage signal which is digitized by microcontroller 5410 and compared to one or more threshold detection values. For example, in some embodiments, threshold detection values are set during a calibration process and can be stored in a memory accessible to microcontroller 5410.

FIG. 56 is an illustrative plot of gap (G) versus proximity (P) for understanding threshold-based proximity detection according to embodiments of the present invention. Gap refers generally to a distance between the testing device and the implant, whereas proximity corresponds to a digitized value of the proximity detection signal. For simplicity, a continuous plot is shown. However, it will be recognized that the digitized proximity value has a finite range determined by the analog-to-digital converter.

As illustrated, at a hypothetical infinite gap (G_{∞}), the proximity value is zero. Since, as a practical matter, analog-to-digital conversions are noise and there will often be environmental disturbances, a first threshold proximity value, Pₙ, is established to serve as a minimum detection value. Proximity values below Pₙ are ignored by the testing device and represent a no-detect state. As the gap between the testing device and the implant decreases, the proximity value increases, first to P₃ and then to P₂.

A maximum proximity value, Pₘₐₓ, is reached at gap Gₘᵢₙ and corresponds to a precise alignment of the testing device and the implant. It is at this location that accurate measurements can be obtained, for example, with positioning scale 5505 of elongated portion 5304. Beyond the minimum gap, Gₘᵢₙ, an over-modulation condition may occur in which the electromagnetic field of resonator circuit 5412 is severely disrupted and no longer provides a reliable indication of proximity. In such cases, microcontroller 5410 may declare a fault by, for example, illuminating a red LED and suspending device operation until power/reset switch 5418 is activated.

Threshold values can also be used to perform a check on the pickup coil of an inductively powered implant device. For example, a proximity value (or range of proximity values) corresponding to a properly functioning implant can be stored in a non-volatile or other memory of the testing device. Proximity values corresponding to a malfunctioning implant, such as an implant having a damaged pickup coil, can also be stored in the memory. During testing, a problem is indicated when the measured proximity value equals or exceeds the damage threshold, but does not rise to the level of the properly functioning device.

Although various threshold values have been discussed, it is contemplated that a testing device according to embodiments of the present invention can include more or fewer thresholds, and that threshold values can be readily added or removed. Also, different thresholds can be associated with different operating modes of the testing device. As one example, a single threshold may be used in a presence-detect operating mode whereas several thresholds may be used in a coil-test mode.

Microcontroller 5410 updates status indicator 5422 based on the output of proximity detection circuit 5424. In one embodiment, status indicator 5422 includes two LEDs as well as an audible alert. When the output of proximity detection circuit 5424 indicates that the implant device is not detected, the LEDs are extinguished and the audible alert is disabled. As the testing device approaches the implant, the LEDs flash and the audible alert beeps at a rate which corresponds roughly to proximity. For example, the two LEDs flash alternately and the flash-rate interval decreases with the separation distance. Similarly, the pitch of the audible alert and the beep interval can change with proximity to inform a user of the testing device that the implant is or is not detected at the current location. By manipulating the testing device according to cues from status indicator 5422, the implant can be quickly detected and its precise location can be ascertained.

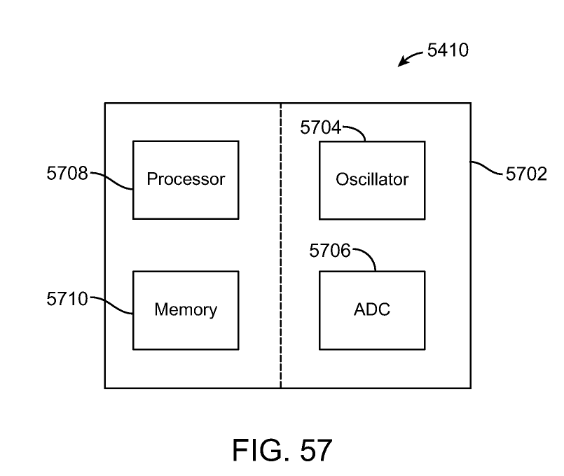

FIG. 57 depicts an exemplary microcontroller 5410 such as can be used with a testing device as described in connection with FIGS. 53-54. As shown, microcontroller 5410 includes embedded peripherals 5702 as well as processor 5708 and memory 5710 elements. Embedded peripherals 5702 include oscillator 5704 and analog-to-digital converter (ADC) 5706. Oscillator 5704 generates programmable drive signals having a frequency that is determined by processor 5708. The drive signals are supplied, for example, to resonator circuit 5412 for controlling its operating frequency and thus its current flow. Analog-to-digital converter 5706 produces a digital value corresponding to the output of proximity detection circuit 5424 and/or the voltage level of battery 5408.

Memory 5710 can include read-only memory (ROM) and random-access memory (RAM) and other forms of volatile and non-volatile storage. In one embodiment, memory 5710 stores programming instructions as well as calibration data. Programming instructions can be loaded through communications interface 5426 and typically include software for controlling operation of the testing device and for communicating with other devices. For example, programming instructions can provide a command interface for exchanging data through communications interface 5426. Calibration data can include values for low battery cut-off, one or more proximity detection thresholds, and a device serial number. A data integrity value can be stored or calculated to verify integrity of the calibration data and programming instructions. If data corruption is detected, microcontroller 5410 can signal a fault and suspend device operation.

In some embodiments, microcontroller 5410 supports a set of runtime commands through communications interface 5426. As shown in the exemplary table, runtime commands can include read-commands for retrieving information from the testing device, write commands for storing data in the testing device, and diagnostic testing commands.

| Command | Response |
|---|---|
| S or s | Print software version |
| c | Print calibration values |
| C | Receive calibration values, then write to memory |
| V | Print ADC value for shunt voltage |
| b | Print ADC value for battery cut-off voltage |
| L or l | LED test - Flash the LEDs |
| f | Perform frequency scan of LC circuit and print results |
| p | Perform frequency scan, but print only peak (resonant) value; |
| | set drive signal frequency to peak value |
| a | Test audio beep |
| Other | Return error message |

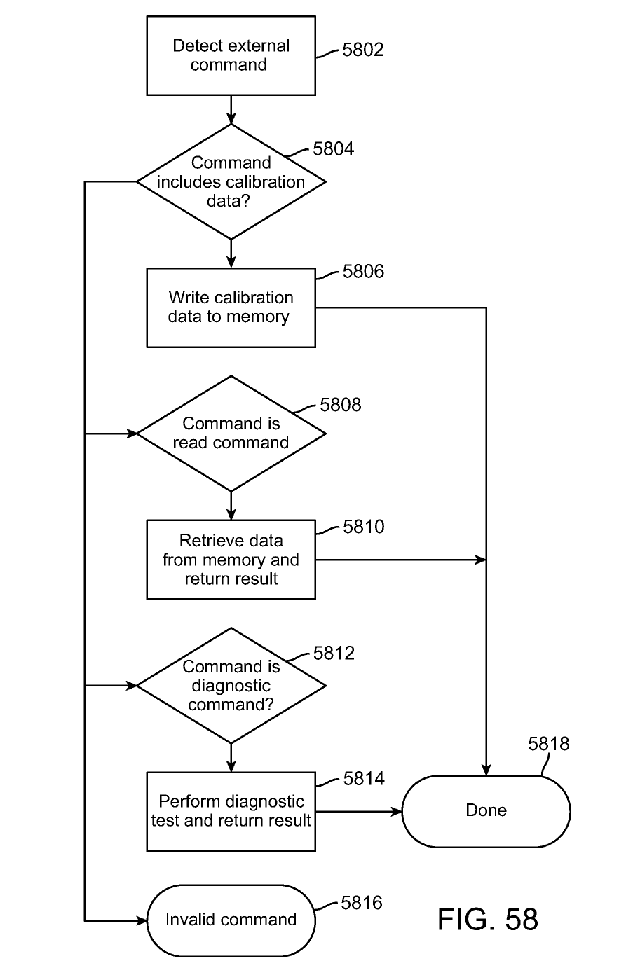

FIG. 58 is a flowchart showing aspects of command processing according to embodiments of the present invention. Commands such as those listed above can be received and executed by a microcontroller or other processor of the testing device. For example, commands can be received through communications interface 5426 for execution by microcontroller 5410. In a preferred embodiment, communication interface 5426 supports serial data exchange with microcontroller 5410.

At block 5802, an external command is detected. In some embodiments, the command can be a read command, a write command, or a diagnostic command. For example, the command ‘c’ is a read command which causes the testing device to output current calibration values. On the other hand, ‘C’ is a write command which can be used to set values of calibration data. Command ‘f’ is an example of a diagnostic command which causes the testing device to perform a frequency scan of the resonator circuit and to output values representative of resonator current at different drive frequencies.

At block 5804, it is determined if the command is a write command. If so, it will include calibration or other data. At block 5806, the data is written to a memory of the testing device and command processing completes at terminal block 5818. If the command is a read command, block 5808, data is retrieved from memory and output through communications port. After returning the requested values, block 5810, processing is complete. The testing device responds to a diagnostic command by performing the requested operations and returning values as appropriate. This is illustrated at blocks 5812-5814. Finally, if the command is not a read command, a write command, or a diagnostic command, it is invalid (block 5816). In this case, the testing device may output an error message and terminate command processing.

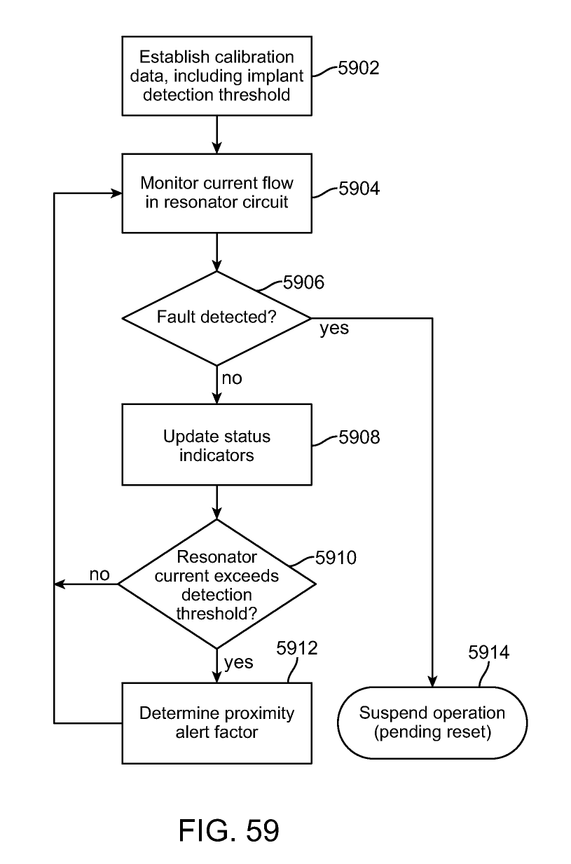

FIG. 59 is a flowchart showing aspects of proximity detection according to embodiments of the present invention. The processing operations described in FIG. 59 can be coordinated, for example, by microcontroller 5410 or a like processing device. At block 5902, calibration data is established at the implant testing device. This can include retrieving calibration data from a memory of the testing device. In one embodiment, the testing device includes FLASH memory (non-volatile) storage and calibration data, including one or more detection thresholds, is read from the memory when the testing device is activated.

At block 5904, the testing device begins monitoring current flow in the resonator circuit. For example, a microcontroller can be configured to sample the value of resonator current at regular intervals. When a fault condition is detected, at block 5906, operation of the testing device is suspended. For example, when an over-modulation condition is detected, the microcontroller may suspend operation, block 5914, pending a reset of the resonator and proximity detection circuits. In some embodiments, fault conditions are signaled by a red LED and/or warning beep.

When a fault is not detected then, at block 5908, status indicators are updated based on a proximity alert factor. While searching for the implant, the proximity alert factor can be set to a no-detect condition and the status indicators can be updated accordingly. Thereafter, the status indicators can be updated according to the proximity alert factor by, for example, adjusting a flash-rate of LEDs and/or the pitch and repeat interval of an audible alert.

At block 5910, it is determined if resonator current exceeds a detection threshold. If it does not, this can indicate that the implant is not detected and the testing device continues to monitor current flow. If resonator current does exceed the detection threshold, at block 5912, the proximity alert factor is determined. For example, the proximity alert factor can provide an indication of how close the testing device is to the implant and thus serves as a basis for updating the status indicators.

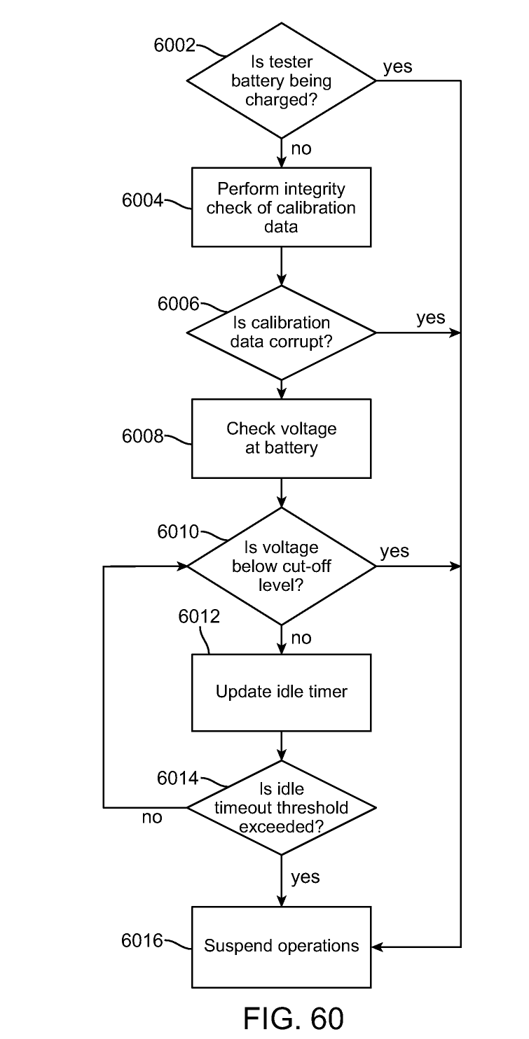

FIG. 60 is a flowchart illustrating aspects of power management according to embodiments of the present invention. At block 6002, it is determined whether a battery of the testing device is being charged. When the battery is charging, device operation is suspended at block 6016. For example, referring again to FIG. 54, power control block 5420 can be configured to open high-side switch 5428 for so long as charge controller 5406 supplies a charging current to battery 5408.

At block 6004, an integrity check is performed on the calibration data. When the calibration data is corrupt, block 6006, device operation is suspended. For example, corrupt calibration data can indicate that the testing device needs reprogramming and may therefore be treated as a fault condition. A check of battery voltage is performed at block 6008. When a low-voltage condition is detected, block 6010, device operation is suspended. For example, microcontroller 5410 can be configured to provide a suspend signal to power control block 5420 when the battery voltage does not exceed a cut-off level included as part of the calibration data.

When battery voltage exceeds the cut-off level, an idle timer is updated (block 6012). The idle timer helps to conserve battery power by monitoring for device inactivity and is reset when the device is actively used. If a timeout period is exceeded, block 6014, then device operation is suspended. Otherwise, processing continues at block 6010. For example, a timeout value of approximately 10 minutes can be established during calibration of the testing device. In that case, after 10 minutes of inactivity, operation of the device is be suspended.

### Method of Using

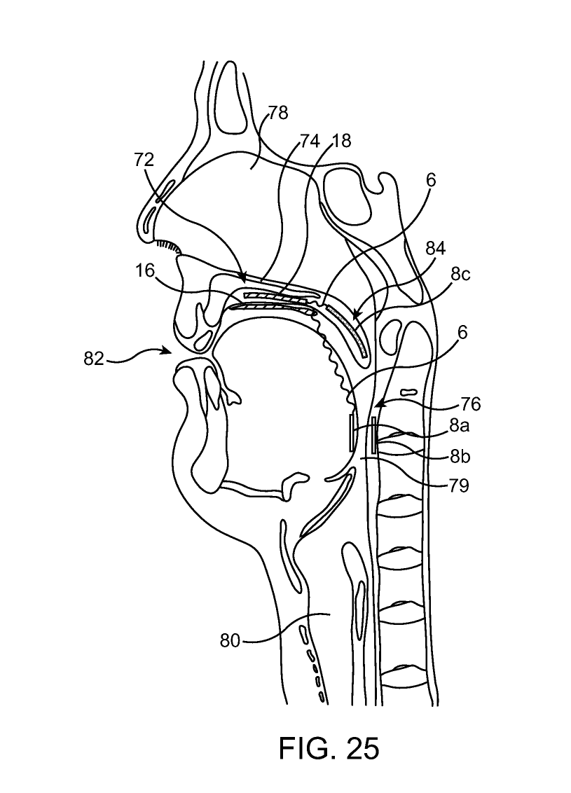

FIG. 25 illustrates an embodiment of a method of the airway implant device of the present invention. In this embodiment, the first inductor 18 is implanted in the mouth roof 72, for example in or adjacent to the hard palate 74. Wire leads 6 connect the first inductor 18 to the actuator elements 8a, 8b, and 8c. A first actuator element 8a is implanted in the base of the tongue at the pharynx wall 76. A second actuator element 8b is integral with the first actuator element 8a (e.g., as two sections of a hollow cylindrical actuator element 8, such as shown in FIG. 17). The first and second actuator elements 8a and 8b can be separate and unattached elements. The third actuator element 8c is implanted in the uvula and/or soft palate 84. The actuator elements 8 can also be implanted in the wall of the nasal passages 78, higher or lower in the pharynx 79, such as in the nasal pharynx, in the wall of the trachea 80, in the larynx (not shown), in any other airway, or combinations thereof. The second inductor 16 is worn by the patient in the mouth 82. The second inductor 16 is connected to an integral or non-integral power source. The second inductor 16 comprises one or multiple induction coils. The second inductor 16 inductively transmits RF energy to the first inductor 18. The first inductor 18 changes the RF energy into electricity. The first inductor 18 sends a charge or current along the wire leads 6 to the actuator elements 8a, 8b, and 8c. The actuator elements 8a, 8b, and 8c are energized by the charge or current. The energized actuator elements 8a, 8b, and 8c increase the stiffness and/or alter the shape of the airways. The energized actuator elements 8a, 8b, and 8c modulate the opening of the airways around which the actuator elements 8a, 8b, and 8c are implanted. The non-energized actuator elements 8a, 8b, and 8c are configured to conform to the airway around which the actuator elements 8a, 8b, and 8c are implanted. The non-energized actuator elements 8a, 8b, and 8c are flexible and soft.

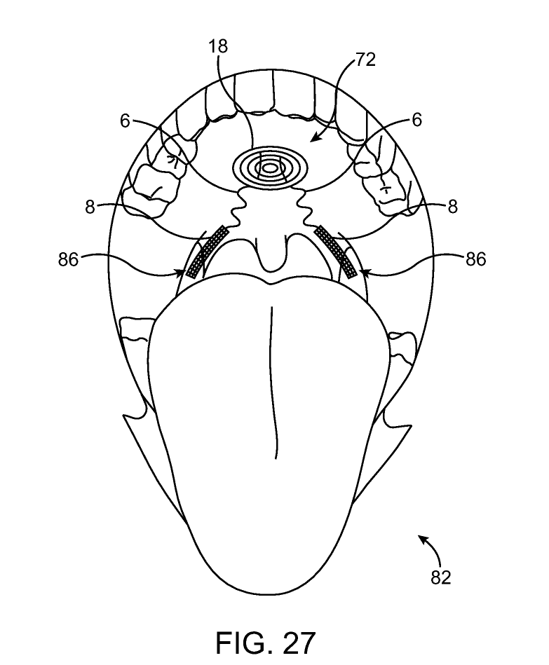

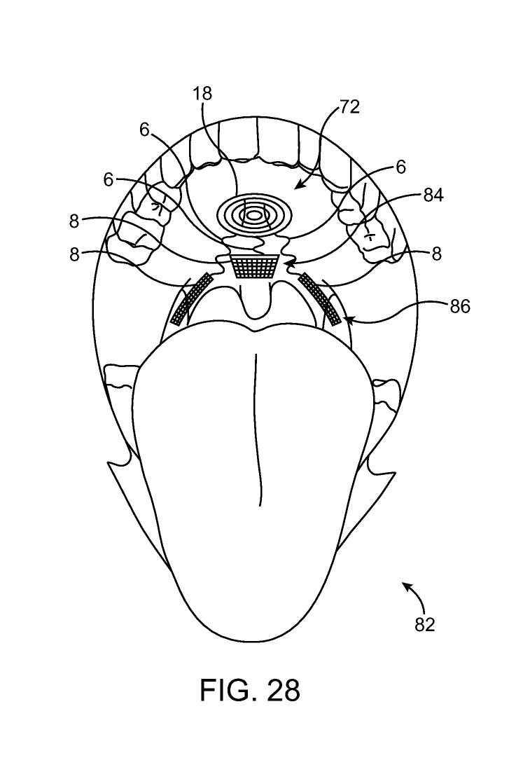

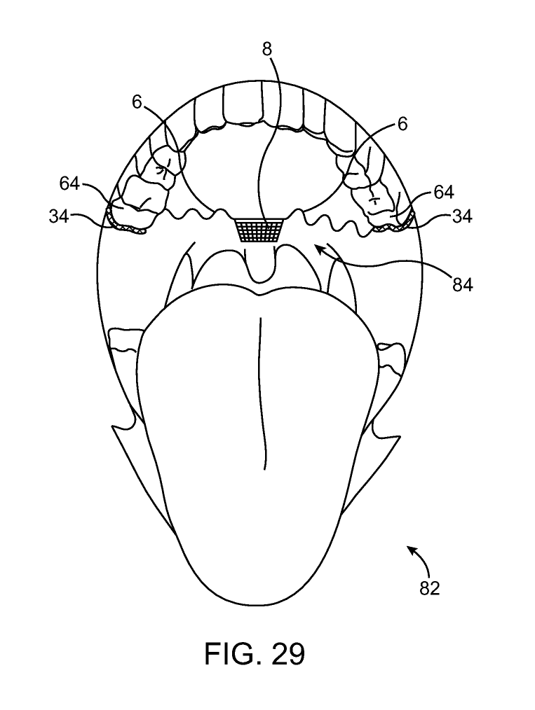

FIG. 26 illustrates another embodiment of the invention. In this embodiment, the first inductor 18 is implanted in the mouth roof 72 and attached to a actuator element 8 via the wire lead 6. The actuator element 8 is preferably in the soft palate 84. In another embodiment, FIG. 27 illustrates that the first inductor 18 is implanted in the mouth roof 72 and attached to two actuator elements 8 via two wire leads 6. The actuator elements 8 are implanted in side walls 86 of the mouth 82. In yet another embodiment, as illustrated in FIG. 28, the first inductor 18 is implanted in the mouth roof 72 and attached to three actuator elements 8 via three wire leads 6. The actuator elements 8 are implanted in the soft palate 84 and the side walls 86 of the mouth 82. FIG. 29 illustrates an embodiment in which the first conductors (not shown, e.g., the tooth sockets), are attached to, and in conductive electrical communication with, the second conductors. The retainer 66, such as shown in FIG. 23, can be worn by the patient to energize the actuator element 8. The tooth sockets are removably attached to the first conductors 34. The first conductors 34 are dental fillings, conductive posts adjacent to and/or through the teeth 64.

FIG. 33 illustrates an embodiment in which a patient 88 has the first transducer (not shown) implanted in the patient's cheek and wears the non-implanted portion 22, such as shown in FIG. 24, on the outside of the patient's cheek. The non-implanted portion 22 energizes the implanted portion (not shown).

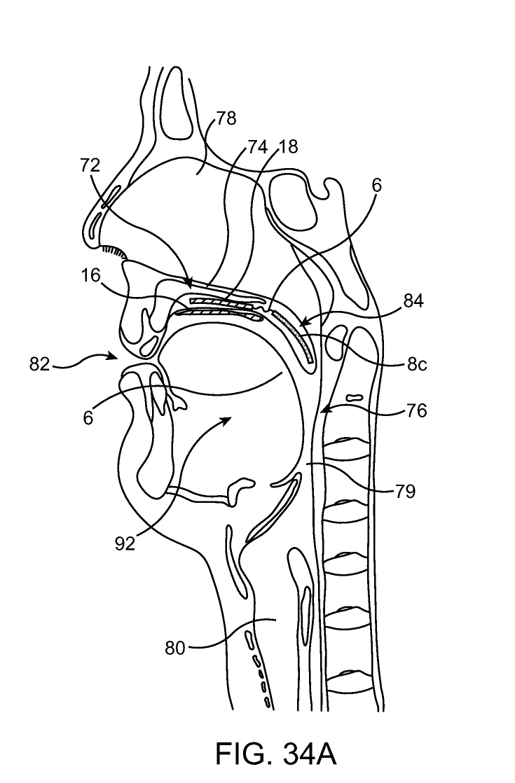

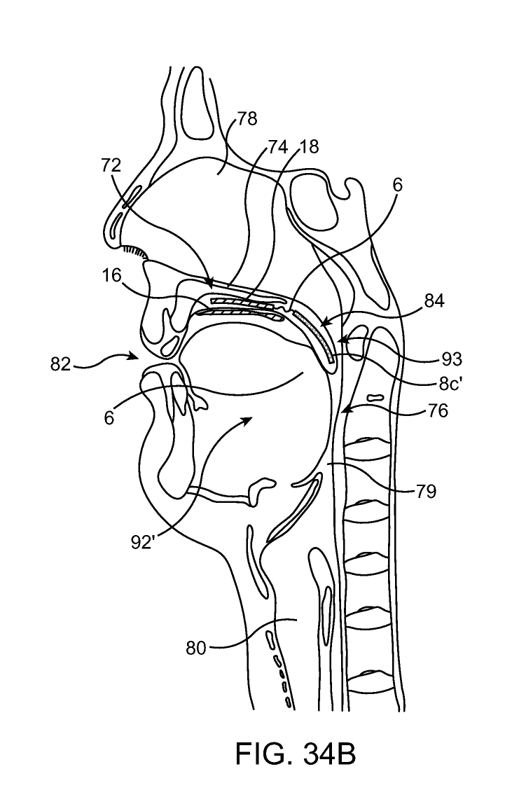

FIGS. 34-36 depict some of the ways in which the implant devices function to open the airways. FIGS. 34A and 34B depict a side view of a patient with a soft palate implant 8c and a non-implanted portion of the device, with a second inductor 16, which in this case is a wearable mouth piece. The wearable mouth piece includes a transmitter coil, a power source, and other electronics, which are not depicted. Also, shown is a first inductor 18. The implant device has the ability to sense and deflect the tongue so as to open the airway. FIG. 34A depicts the tongue 92 in its normal state. During sleep, when the tongue collapses 92′, as shown in FIG. 34B, the actuator element 8c′ senses the collapsed tongue and is energized via the mouthpiece and first inductor and it stiffens to push away the tongue from the airway and keeps the airway open. This opening of the airway can be partial or complete. In some embodiments, particularly the embodiments without the sensor, the implant is powered when the patient is asleep such that the actuator element 8 is energized and keeps the collapsed tongue away from the airway.

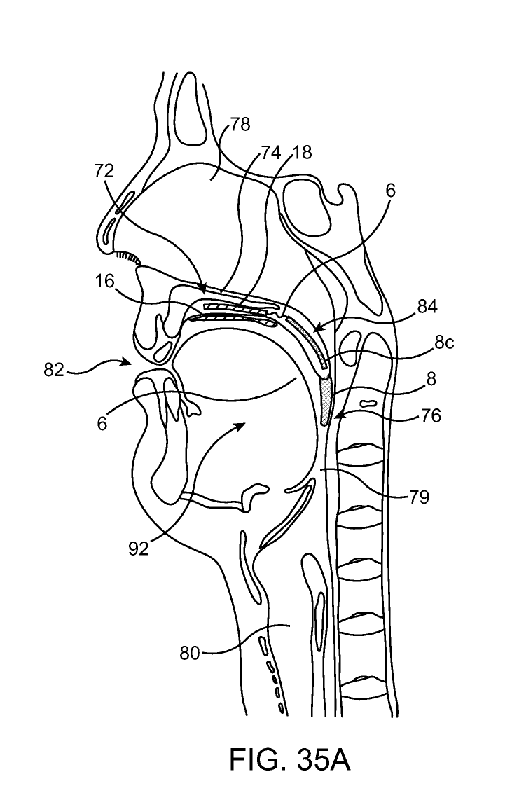

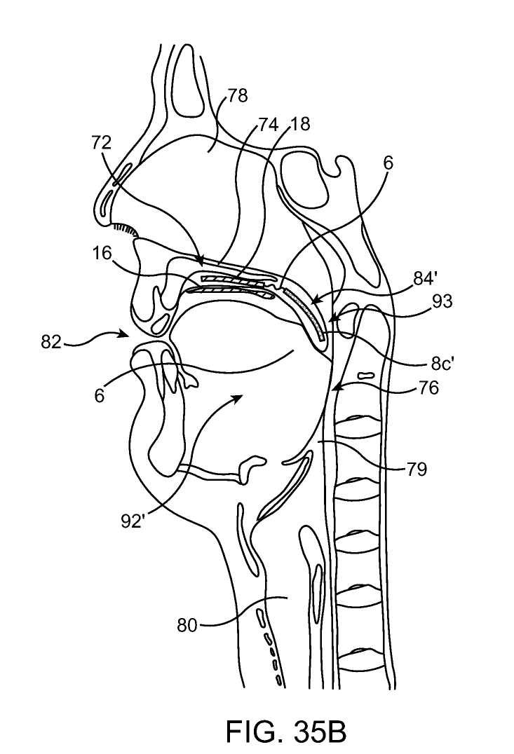

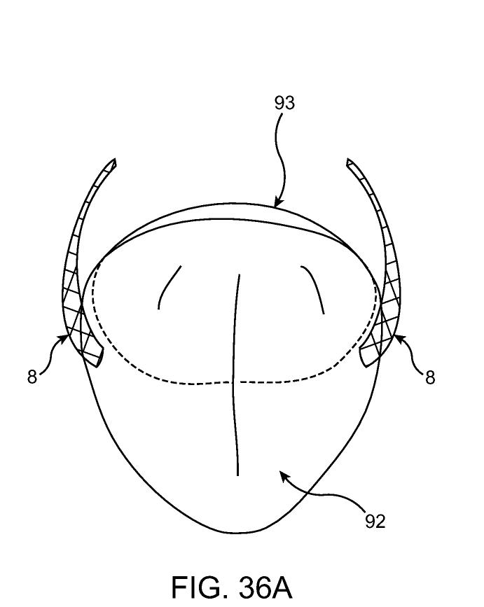

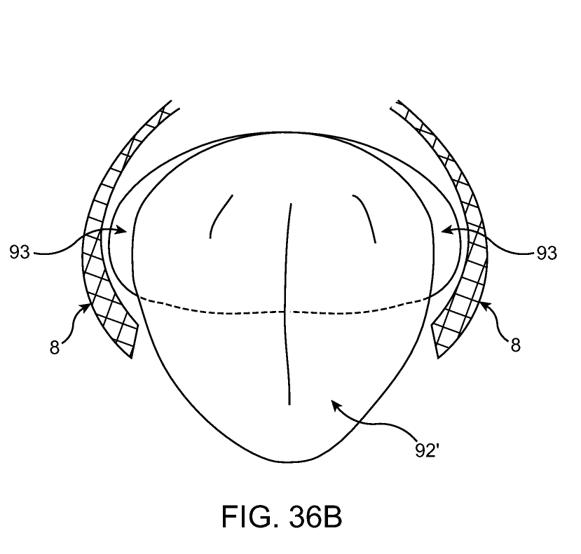

FIGS. 35 and 36 depict an embodiment of keeping the airways open with lateral wall implants. FIG. 35A shows a side view of a patient's face with a actuator element 8 located in the lateral wall of the airway. FIG. 35A depicts the tongue 92 in its normal state. FIG. 35B depicts the tongue 92′ in a collapsed state. When the tongue is in this state or before it goes into the collapsed state the actuator element 8 is energized so as to stretch the lateral walls and open the airway, as shown in FIG. 36B. FIGS. 36A and 36B are a view of the airway as seen through the mouth of patient. FIG. 36 A depicts the actuator elements 8 in a non-energized state and the tongue in a non-collapsed state. When the tongue collapses or it has a tendency to collapse, such as during sleep, the actuator element 8 is energized and airway walls are pushed away from the tongue and creates an open air passageway 93. This embodiment is particularly useful in obese patients.

### Airway Diseases

During sleep, the muscles in the roof of the mouth (soft palate), tongue and throat relax. If the tissues in the throat relax enough, they vibrate and may partially obstruct the airway. The more narrowed the airway, the more forceful the airflow becomes. Tissue vibration increases, and snoring grows louder. Having a low, thick soft palate or enlarged tonsils or tissues in the back of the throat (adenoids) can narrow the airway. Likewise, if the triangular piece of tissue hanging from the soft palate (uvula) is elongated, airflow can be obstructed and vibration increased. Being overweight contributes to narrowing of throat tissues. Chronic nasal congestion or a crooked partition between the nostrils (deviated nasal septum) may be to blame.

Snoring may also be associated with sleep apnea. In this serious condition, excessive sagging of throat tissues causes your airway to collapse, preventing breathing. Sleep apnea generally breaks up loud snoring with 10 seconds or more of silence. Eventually, the lack of oxygen and an increase in carbon dioxide signal causes the person to wake up, forcing the airway open with a loud snort.

Obstructive sleep apnea occurs when the muscles in the back of the throat relax. These muscles support the soft palate, uvula, tonsils and tongue. When the muscles relax, the airway is narrowed or closed during breathing in, and breathing is momentarily cut off. This lowers the level of oxygen in the blood. The brain senses this decrease and briefly rouses the person from sleep so that the airway can be reopened. Typically, this awakening is so brief that it cannot be remembered. Central sleep apnea, which is far less common, occurs when the brain fails to transmit signals to the breathing muscles.

Thus, it can be seen that airway disorders, such as sleep apnea and snoring, are caused by improper opening of the airway passageways. The devices and methods described herein are suitable for the treatment of disorders caused by the improper opening of the air passageways. The devices can be implanted in any suitable location such as to open up the airways. The opening of the passageways need not be a complete opening and in some conditions a partial opening is sufficient to treat the disorder.

In addition to air passageway disorders, the implants disclosed herein are suitable for use in other disorders. The disorders treated with the devices include those that are caused by improper opening and/or closing of passageways in the body, such as various locations of the gastro-intestinal tract or blood vessels. The implantation of the devices are suitable for supporting walls of passageways The devices can be implanted in the walls of the gastro-intestinal tract, such as the esophagus to treat acid reflux. The gastro-intestinal tract or blood vessel devices can be used in combination with the sensors described above. Also, the implants and/or sphincters can be used for disorders of fecal and urinary sphincters. Further, the implants of said invention can be tailored for specific patient needs.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

What is claimed is:

1. A handheld testing device for detecting the presence of an airway implant unit within a patient's body, comprising:
a body having an elongated portion adapted for insertion into the patient's mouth and a handle for manipulating the testing device;
a detector disposed within the body comprising a resonator circuit and a processor, wherein the processor is configured to monitor the resonator circuit and to detect a proximity of the implant unit to the testing device; and
one or more status indicators coupled to the processor and configured to signal the proximity of the implant unit to the testing device.

2. The handheld testing device of claim 1 wherein the detector is disposed at a distal end of the elongated portion, and wherein the processor is disposed in the handle portion of the body.

3. The handheld testing device of claim 1 wherein the elongated portion comprises a scale indicative of a position of the detector within the patient's mouth.

4. The handheld testing device of claim 1 wherein the body comprises a top surface and a bottom surface, and wherein the one or more status indicators is disposed on the top and bottom surfaces so as to be visible by a physician when the elongated portion is within the patient's mouth and the physician's direction of view is changed from top surface viewing to bottom surface viewing.

5. The handheld testing device of claim 1 wherein the resonator circuit comprises an inductor and a capacitor, and wherein the processor is configured to deliver a drive signal to the resonator circuit.

6. The handheld testing device of claim 5 wherein the processor is configured to detect the proximity of the implant unit to the testing device based on a change in resonator current.

7. The handheld testing device of 6 wherein the processor adjusts an output signal to the one or more status indicators based on detecting the proximity of the implant unit.

8. The handheld testing device of claim 1 wherein the one or more status indicators comprise light emitting diodes.

9. The handheld testing device of claim 1 wherein the one or more status indicators comprise an audible tone.

10. The handheld testing device of claim 1 further comprising a battery configured to power the processor and the resonator circuit.

11. The handheld testing device of claim 10 further comprising a charge control circuit, wherein the charge control circuit is configured to deliver a charging current to the battery.

12. The handheld testing device of claim 1 further comprising a control interface, and wherein the processor is configured to communicate with an external device using the control interface.

13. The handheld testing device of claim 12 wherein the processor communicates a status of the handheld testing device in response to command received through the control interface.

14. The handheld testing device of claim 12 wherein the control interface is adapted for serial communications with the external device.

15. The handheld testing device of claim 12 further comprising a non-volatile memory.

16. The handheld testing device of claim 15 wherein the non-volatile memory is configured to store calibration data for the testing device.

17. The handheld testing device of claim 15 wherein the non-volatile memory is configured to store program code executed by the processor.

18. A method of detecting a palatal implant, the method comprising:
generating an electromagnetic field at a testing device;
detecting a variation in the electromagnetic field due to a proximity of the testing device to the palatal implant; and
indicating the proximity of the testing device to the palatal implant based on the variation of the electromagnetic field.

19. The method of claim 18 wherein generating the electromagnetic field comprises driving an inductor-capacitor (LC) circuit at approximately a resonant frequency of the inductor-capacitor circuit.

20. The method of claim 18 further comprising storing calibration data within a non-volatile memory of the testing device.

21. The method of claim 19 wherein detecting a variation in the electromagnetic field comprises detecting a change in the electromagnetic field of the inductor element.

22. The method of claim 18 wherein indicating the proximity of the testing device to the palatal implant comprises generating an audible tone.

23. The method of claim 22 further comprising varying a frequency of the audible tone based on the proximity of the testing device to the implant.

24. The method of claim 18 wherein indicating the proximity of the testing device to the palatal implant comprises providing at least one visual indicator.

25. The method of claim 24 further comprising varying a flash rate of the at least one visual indicator based on the proximity of the testing device to the implant.

26. The method of claim 18 further comprising providing a status of the testing device in response to at least one external command.

27. The method of claim 18 further comprising determining a location of the testing device within the patient's mouth using a positioning scale.

28. A handheld testing device for detecting the presence of a palatal implant unit within a patient's body, comprising:
a body having an elongated portion adapted for insertion into the patient's mouth and a handle portion adapted for manipulating the testing device;
a detector disposed at a distal end of the elongated portion and comprising a resonator circuit configured to generate an electromagnetic field;
a processor disposed within the handle and configured to detect a proximity of the testing device to the implant unit based upon the electromagnetic field and to generate an output signal indicative of the proximity; and
a user interface circuit including at least one light emitting diode (LED), the user interface circuit configured to drive the at least one LED based on the output signal.

29. The handheld device of claim 28 wherein the user interface circuit further comprises an audio generator configured to vary a pitch of an audible signal based on the output signal.

30. The handheld device of claim 28 wherein the resonator circuit comprises an inductor-capacitor (LC) circuit, and wherein the processor is configured to drive the LC circuit at approximately a resonant frequency of the LC circuit.

31. The handheld device of claim 30 further comprising an analog-to-digital converter (ADC) configured to measure a current of the LC circuit, and wherein the processor is configured to detect the proximity of the testing device to the implant based upon an output of the ADC.

32. The handheld device of claim 28 wherein the user interface varies a flash-rate of the at least one LED based on the output signal.

33. A handheld device used to detect an implanted medical prosthesis in a patient's body comprising:
a detector comprising a transmit circuit and a processor, wherein the processor is configured to monitor the transmit circuit and to detect a proximity of the implant unit to the testing device; and
an indicator coupled to the processor and configured to signal the proximity of the implant unit to the testing device.

34. The handheld device of claim 33 further comprising a processor and software to upload the data collected by the implanted prosthesis.

35. The handheld device of claim 33 further comprising a processor and software to download data to the implanted prosthesis.
